(19) United States
(12) Patent Application Publication
(10) Pub. No.: US 2021/0161975 A1
(43) Pub. Date: Jun. 3, 2021

(54) INACTIVATED BACILLUS COAGULANTS AND USES THEREOF FOR REDUCING STRESS

(71) Applicant: Ganeden Biotech, Inc., Mayfield Heights, OH (US)

(72) Inventors: Sean Farmer, Miami, FL (US); David Keller, Beachwood, OH (US); Howard Cash, Mentor, OH (US)

(21) Appl. No.: 16/641,622

(22) PCT Filed: Oct. 5, 2018
(86) PCT No.: PCT/US18/54705

§ 371 Date: Feb. 24, 2020

Related U.S. Application Data
(60) Provisional application No. 62/568,764, filed on Oct. 5, 2017.

(51) Int. Cl. A61K 35/742; A61P 25/22; A61P 25/24; A61P 3/08

(52) U.S. Cl. CPC A61K 35/742; A61P 25/22; A61K 2035/115; A61P 3/08; A61P 25/24

(57) ABSTRACT

The present subject matter provides compositions and methods comprising inactivated, non-viable, or dead Bacillus coagulans bacteria for the prevention or treatment of stress.

### CROSS REFERENCE TO RELATED APPLICATIONS

This application claims the benefit of U.S. Provisional Application 62/568,764 filed on Oct. 5, 2017, the entire contents of which is incorporated herein by reference in its entirety.

### BACKGROUND

The gastrointestinal microflora plays a number of vital roles in maintaining gastrointestinal tract function and overall physiological health. The growth and metabolism of the many individual bacterial species inhabiting the gastrointestinal tract depend primarily upon the substrates available to them, most of which are derived from the diet. These findings have led to attempts to modify the composition and metabolic activities of the bacterial community through diet, primarily with probiotics, which are live microbial food supplements.

Probiotic organisms are non-pathogenic, non-toxigenic, retain viability during storage, and typically survive passage through the stomach and small intestine. Since probiotics do not generally permanently colonize the host, they need to be ingested regularly for health promoting properties to persist.

### SUMMARY OF THE INVENTION

Provided herein, inter alia, are compositions and methods for treating stress comprising inactivated, non-viable, and/or dead Bacillus coagulans bacteria.

In an aspect, included herein is a method for treating or preventing stress or anxiety in a subject, comprising administering a composition comprising inactivated, non-viable, or dead Bacillus coagulans bacteria to the subject.

In various embodiments, the composition is administered to the subject once per day.

In some embodiments, the composition is administered in the morning, with breakfast, or before breakfast.

In certain embodiments, the composition is administered in the evening, with dinner, or after dinner.

In various embodiments, the effective amount reduces the level of cortisol in the subject. In some embodiments, the level of cortisol is reduced by at least 10%, 20%, 30%, or 40%. In certain embodiments, the level of cortisol is reduced by about 10-40%, about 20-30%, or about 10-30%. In various embodiments, the level of cortisol is reduced from about 5%, 10%, or 15% to about 25%, 30%, or 35%. In some embodiments, the level of cortisol is reduced in urine, blood, or serum of the subject.

In certain embodiments, the subject has been or is characterized, e.g., self described or has been diagnosed as experiencing stress and/or anxiety, due to any one or more of the following events, experiences or scenarios. In certain embodiments, the subject has experienced trauma. In various embodiments, the trauma is emotional trauma or physiological trauma. In certain embodiments, the subject has a mood disorder. In various embodiments, the subject has bipolar disorder. In some embodiments, the subject has anxiety or depression. In certain embodiments, the depression is seasonal depression. In various embodiments, the subject has hypoglycemia. In some embodiments, the subject has post-traumatic stress disorder. In certain embodiments, the subject has chronic pain. In various embodiments, the subject is a soldier or a first responder (e.g., a paramedic, a police officer, or a firefighter). In some embodiments, the subject does not have an infection.

In certain embodiments, the composition is in the form of a tablet, a capsule, a powder, a suspension, an aqueous solution, a food, or a beverage. In various embodiments, the effective amount is less than 50 mg per day. In some embodiments, the effective amount is less than 50 mg, 45 mg, 40 mg, 35 mg, 30 mg, 25 mg, 20 mg, 15 mg, or 10 mg per day. In certain embodiments, the effective amount is from about 10 mg, 15 mg, or 20 mg to about 50, 75, or 100 mg. In various embodiments, the effective amount is from 0.1-1 mg, 0.1-5 mg, 0.1-10 mg, 1-10 mg, 10-50 mg, 50-100 mg, 50-500 mg, 10-150 mg, 100-250 mg, 100-500 mg, 100-750 mg, 250-500 mg, 250-750 mg, 500-750 mg, or 500-1000 mg.

As used herein, an “inactivated” Bacillus coagulans bacterium is a Bacillus coagulans bacterium with reduced internal metabolic activity and reproductive ability. In various embodiments, an inactivated Bacillus coagulans bacterium comprises an intact cell wall. In some embodiments, an inactivated Bacillus coagulans bacterium comprises an intact cell membrane. In some embodiments, an inactivated Bacillus coagulans bacterium comprises a genome that does not have a double strand break.

As used herein, a “non-viable” Bacillus coagulans bacterium is a Bacillus coagulans bacterium with no reproductive ability. In various embodiments, a non-viable Bacillus coagulans bacterium comprises no metabolic activity. In some embodiments, a non-viable Bacillus coagulans vegetative bacterium does not consume or metabolize glucose (e.g., is incapable of using glucose for energy or as a carbon source). In certain embodiments, a non-viable Bacillus coagulans vegetative bacterium is incapable of germination. In various embodiments, the biomass of non-viable Bacillus coagulans bacteria does not change when it is incubated in a Bacillus coagulans growth medium. In some embodiments, a non-viable Bacillus coagulans bacterium comprises a genome with at least one double strand break.

As used herein, a “dead” Bacillus coagulans bacterium is a Bacillus coagulans bacterium that does not have a fully intact cell wall or spore. For example, the cell wall of a dead vegetative Bacillus coagulans bacterium may have one or more structural defects (e.g., fractures, holes, voids, perforations, etc.) that permits fluid to flow freely in and out of the cell. In some embodiments, the cell or spore may be a whole dead/non-viable vegetative cell. In certain embodiments, the cell is non-viable/non-proliferative, yet remains largely structurally (e.g., in terms of the cell wall or spore structure) intact. In various embodiments, a dead Bacillus coagulans bacterium is a fragment of a Bacillus coagulans bacterium that comprises a portion of a Bacillus coagulans cell wall that comprises peptidoglycan and/or lipoteichoic acid. In various embodiments, a dead Bacillus coagulans bacterium comprises a genome that comprises two or more double strand breaks, and the genome is within a cell wall. In some embodiments, a dead Bacillus coagulans bacterium comprises a genome that comprises two or more double strand breaks, and the genome is within a cell wall. In certain embodiments, a dead Bacillus coagulans bacterium comprises 1 or more genome fragments that is at least about 100 kilobases in length (e.g., 1, 2, 3, 4, or 5 at least about 200, 300, 400, 500 kilobases in length) within a cell wall.

In various embodiments, inactivated, non-viable, or dead Bacillus coagulans bacteria and/or particles retain the presence of undamaged lipoteichoic acid. For example, the lipoteichoic acid comprises immune-activating activity.

In some embodiments, provided herein are particles that comprise, consist essentially of, or consist of inactivated, non-viable, and/or dead Bacillus coagulans bacteria. In various embodiments, a particle may comprise inactivated, non-viable, and/or dead Bacillus coagulans vegetative cells and/or fragments thereof. In certain embodiments, the particle is greater than 5 μm in at least one dimension. For example, the particle may comprise at least one dimension of about 5 μm, 10 μm, 25 μm, 50 μm, 100 μm, 150 μm or 200 μm, to about 300 μm, 350 μm, 400 μm, 450 μm, 500 μm, 750 μm, or 1000 μm. In some embodiments, a particle comprises a length of 5-1000 μm (e.g., a length of about 5 μm, 10 μm, 25 μm, 50 μm, 100 μm, 150 μm or 200 μm to about 300 μm, 350 μm, 400 μm, 450 μm, 500 μm, 750 μm, or 1000 μm) and a width of 5-1000 μm (e.g., a width of about 5 μm, 10 μm, 25 μm, 50 μm, 100 μm, 150 μm, or 200 μm, to about 300 μm, 350 μm, 400 μm 450 μm, 500 μm, 750 μm, or 1000 μm). In certain embodiments, the particle comprises a diameter of 5-1000 μm (e.g., about 5 μm, 10 μm, 25 μm, 50 μm 100 μm, 150 μm 200 μm, 250 μm, 300 μm, 350 μm, 400 μm, 450 μm, 500 μm, 750 μm or 1000 μm). Compositions containing purified compounds from Bacillus coagulans cell walls are also included.

Also provided are methods of administering such inactivated, non-viable, or dead Bacillus coagulans bacteria, particles (e.g., comprising inactivated, non-viable, and/or dead Bacillus coagulans bacteria), and compounds (such as lipoteichoic acid or peptidoglycan), e.g., to treat (e.g., reduce) or prepare for (e.g., prevent) stress.

In an aspect, provided herein is a composition comprising inactivated, non-viable, or dead Bacillus coagulans bacteria and/or particles comprising such bacteria. In various embodiments, the inactivated, non-viable, or dead Bacillus coagulans bacteria and/or particles are present in an amount that reduces the level of cortisol (e.g., in the blood or serum) in a subject. In some embodiments, the inactivated, non-viable, or dead Bacillus coagulans bacteria and/or particles are present in an amount that prevents or reduces stress or anxiety in a subject.

In various embodiments, the inactivated, non-viable, or dead Bacillus coagulans bacteria (or particles comprising such bacteria) comprise inactivated, non-viable, or dead Bacillus coagulans vegetative bacteria. In some embodiments, the inactivated, non-viable, or dead Bacillus coagulans bacteria or particles comprise inactivated, non-viable, or dead Bacillus coagulans vegetative bacteria and spores. In certain embodiments, the inactivated, non-viable, and/or dead Bacillus coagulans bacteria (or particles comprising such bacteria) are dried, e.g., contain less than about 10%, 5%, 1%, 0.1%, 0.01%, 0.001%, 0.0001%, or 0.00001% water moisture by weight. In various embodiments, the inactivated, non-viable, and/or dead Bacillus coagulans bacteria (or particles comprising such bacteria) have a water activity of less than about 5, 3, 4, 2, 1.5, 1, 0.75, 0.5, 0.25, or 0.1. As used herein, the term “water activity” is the vapor pressure of water in a substance (e.g. a composition such as particles comprising inactivated, non-viable, and/or dead Bacillus coagulans), divided by the vapor pressure of distilled water at the same temperature. Water activity is often represented by the mathematical equation a_{w}=p/p0, where p is the vapor pressure of water in the substance, and p0 is the vapor pressure of distilled water at the same temperature. Using this particular definition, distilled water has a water activity of 1. The water activities expressed herein are at a temperature of 25° C.

In various embodiments, the composition further comprises an excipient or carrier.

In some embodiments, the composition further comprises maltodextrin, inulin, inositol, trehalose, microcrystalline cellulose (MCC), calcium lactate, magnesium stearate, fructo-oligosaccharide (FOS), or gluco-oligosaccharide (GOS).

In certain embodiments, the inactivated, non-viable, or dead Bacillus coagulans bacteria have been dried, e.g., lyophilized. In some embodiments, particles comprising inactivated, non-viable, or dead Bacillus coagulans bacteria may then be combined with an aqueous solution prior to administration to a human or non-human animal subject. In certain embodiments, the inactivated, non-viable, or dead Bacillus coagulans bacteria have been lyophilized and then combined with an aqueous solution.

In various embodiments, the composition further comprises a surfactant or an emulsifier. In some embodiments, the surfactant comprises polysorbate 20 and/or polysorbate 80. Additional non-limiting examples of surfactants include lecithin, monoglycerides, sorbitan esters, ethoxylates of sorbitan esters, sucrose esters, glycolipids, fatty alcohols, fatty acids, benzalkonium chloride, cetylpyridinium chloride, sodium dodecyl benzenesulfonate, polyethoxylated octyl phenol, N-dodecyl piridinium chloride, lauryl mono-ethanol, sorbitan monoester, dimethyl ether of tetradecyl phosphonic, glycerol diester, dodecyl betaine, anionic surfactants, cationic surfactants, nonionic surfactants, zwetterionic sufactants, and gemini surfactants.

Compositions containing (e.g., comprising, consisting essentially of, or consisting of) Bacillus coagulans peptidoglycan and/or lipoteichoic acid are also provided. In various embodiments, the composition comprises both peptidoglycan and lipoteichoic acid. In some embodiments, the peptidoglycan and/or lipoteichoic acid is purified peptidoglycan and/or lipoteichoic acid. In certain embodiments, the composition does not comprise a viable Bacillus coagulans bacterium.

In some embodiments, the composition comprises a food or beverage composition. In certain embodiments, the composition comprises tea, coffee, and/or an alcoholic beverage. In various embodiments, the composition comprises a fermented food or beverage. In some embodiments, the composition comprises a grain-based composition. In certain embodiments, the composition comprises a baked composition. In various embodiments, the composition comprises a confection. In some embodiments, the composition comprises an omega-3 fatty acid. Omega-3 fatty acids (also called ω-3 fatty acids or n-3 fatty acids) are polyunsaturated fatty acids with a double bond (C═C) at the third carbon atom from the end of the carbon chain. Non-limiting examples of omega-3 fatty acids include hexadecatrienoic acid (HTA), α-Linolenic acid (ALA), stearidonic acid (SDA), eicosatrienoic acid (ETE), eicosatetraenoic acid (ETA), eicosapentaenoic acid (EPA), heneicosapentaenoic acid (HPA), docosapentaenoic acid (DPA), docosahexaenoic acid (DHA), tetracosapentaenoic acid, and tetracosahexaenoic acid. In certain embodiments, the composition comprises a dairy composition. In various embodiments, the composition comprises a non-dairy milk-like composition. In some embodiments, the composition comprises a sports nutrition composition. In certain embodiments, the composition comprises animal feed. In some embodiments, the animal feed comprises feed for a work animal, a companion animal, livestock, or aquaculture.

In certain embodiments, a composition provided herein does not comprise an omega-3 fatty acid. In some embodiments, a composition may comprise an omega-3 fatty acid, but does not comprise docosahexaenoic acid. In various embodiments, a composition may comprise an omega-3 fatty acid, but does not comprise eicosapentaenoic acid. In certain embodiments, a composition may comprise an omega-3 fatty acid, but does not comprise docosahexaenoic acid or eicosapentaenoic acid.

In some embodiments, the subject is an athlete (e.g., a runner, bicyclist, baseball player, soccer player, football player, hockey player, basketball player, or cricket player). In certain embodiments, the subject is a law enforcement officer. In various embodiments, the subject is a firefighter. In some embodiments, the subject is an astronaut. In certain embodiments, the subject is a construction worker. In various embodiments, the subject is a member of an armed force (such as a soldier, a marine, a sailor, or a pilot).

In some embodiments, the animal is a performance animal (such as a military dog, a police dog, a race dog, a show dog, a military horse, a police horse, a race horse, a polo horse, or a show horse), a companion animal (such as a dog or a cat), or a work animal (such as a yak, camel, horse, ox, or yak). In certain embodiments, the subject is a reptile, amphibian, bird, or mammal. In various embodiments, the subject is a primate (such as an ape, monkey, gorilla, orangutan, chimpanzee, or human). In some embodiments, the subject is a parrot, chicken, goose, duck, dog, cat, rabbit, pig, or horse. In certain embodiments, the subject is a ruminant such as a cow, sheep, goat, buffalo, yak, deer, elk, giraffe, or camel. In various embodiments, the animal is a pseudoruminant, such as a hippopotamus. Ruminants have four-chambered stomachs whereas pseudoruminants have three-chambered stomachs. In some embodiments, the animal is a performance animal such as a military dog, a police dog, a race dog, a show dog, a military horse, a police horse, a race horse, a polo horse, or a show horse.

In various embodiments, a subject does not have (e.g., has not been diagnosed with) a gastrointestinal disease or an inflammatory bowel condition. In some embodiments, a subject does not have (e.g., has not been diagnosed with) an infection (e.g., a viral, fungal, parasitic or bacterial infection). In certain embodiments, the subject does not have a viral respiratory infection. In various embodiments, the subject does not have a gastrointestinal infection. Alternatively, the subject is characterized by one or more of the above-described conditions, diseases or infections as well as being characterized as comprising a stress-related symptom or condition. Symptoms, for example, increased cortisol level, high blood pressure, elevated heart rate and the like.

In some embodiments, the inactivated, non-viable, or dead Bacillus coagulans bacteria comprise between about 0.000001% to about 10% by weight of the composition. In certain embodiments, a composition provided herein may be about 0.000001%, 0.00001%, 0.0001%, 0.001%, 0.01%, 0.1%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10% inactivated, non-viable, or dead Bacillus coagulans bacteria by weight. In various embodiments, a composition provided herein may be at least 0.000001%, 0.00001%, 0.0001%, 0.001%, 0.01%, 0.1%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10% inactivated, non-viable, or dead Bacillus coagulans bacteria by weight. In some embodiments, a composition provided herein may be less than 0.000001%, 0.00001%, 0.0001%, 0.001%, 0.01%, 0.1%, 1%, 1.5% 2%, 2.5%, 3%, 3.5%, 4%, 4.5%, 5%, 5.5%, 6%, 6.5%, 7%, 7.5%, 8%, 8.5%, 9%, 9.5%, or 10% dead or inactivated Bacillus coagulans bacteria by weight. In certain embodiments, a composition comprises at least 0.000001%, 0.00001%, 0.0001%, 0.001%, 0.01%, 0.1% but less than 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10% inactivated, non-viable, or dead Bacillus coagulans bacteria by weight.

In various embodiments relating to particles, the number of particles in a composition is about 1×10³-1×10²⁰, 1×10³-1×10⁹, 1×10³-1×10⁶, 1×10⁶-1×10⁹, 1×10⁶-1×10²⁰, 1×10⁶-1×10¹⁵, 1×10⁶-1×10¹², 1×10⁸-1×10¹⁷, 1×10¹⁰-1×10²⁰, 1×10³-1×10¹⁰, 1×10⁵-1×10¹⁰, 1×10⁵-1×10¹⁵, 1×10¹⁰-1×10¹⁵, 1×10¹⁴-1×10¹⁶, or about, at least, or less than 5, 10, 50, 100, 500, 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴ 1×10¹⁵, 1×10¹⁶, 1×10¹⁷, 1×10¹⁸, 1×10¹⁹, or 1×10²⁰ particles. In some embodiments, the number of particles in a composition is about 1×10³-1×10²⁰, 1×10³-1×10⁹, 1×10³-1×10⁶, 1×10⁶-1×10⁹, 1×10⁶-1×10²⁰, 1×10⁶-1×10¹⁵, 1×10⁶-1×10¹², 1×10⁸-1×10¹⁷, 1×10¹⁰-1×10²⁰, 1×10³-1×10¹⁰, 1×10⁵-1×10¹⁰, 1×10⁵-1×10¹⁵, 1×10¹⁰-1×10¹⁵, 1×10¹⁴-1×10¹⁶ per gram or about, at least, or less than 5, 10, 50, 100, 500, 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, or 1×10²⁰ particles per gram. In certain embodiments, the number of particles in a composition is about 1×10⁹-1×10¹¹ per gram or about 1×10⁹, 1.5×10⁹, 2×10⁹, 2.5×10⁹, 3×10⁹, 3.5×10⁹, 4×10⁹, 4.5×10⁹, 5×10⁹, 5.5×10⁹, 6×10⁹, 6.5×10⁹, 7×10⁹, 7.5×10⁹, 8×10⁹, 8.5×10⁹, 9×10⁹, 9.5×10⁹, 1×10¹⁰, 1.5×10¹⁰, 2×10¹⁰, 2.5×10¹⁰, 3×1010, 3.5×10¹⁰, 4×10¹⁰, 4.5×10¹⁰, 5×10¹⁰, 5.5×10¹⁰, 6×10¹⁰, 6.5×10¹⁰, 7×10¹⁰, 7.5×10¹⁰, 8×10¹⁰, 8.5×10¹⁰, 9×10¹⁰, 9.5×10¹⁰, or 1×10¹¹, particles per gram.

In embodiments, Bacillus coagulans bacteria are purified (e.g., from media or supernatant in a growth culture) before being deactivated (e.g., killed). With respect to a Bacillus coagulans bacterium, the terms “purified” and “substantially purified” mean a Bacillus coagulans bacterium that is substantially free of contaminating microorganisms or other macromolecules, e.g., polysaccharides, nucleic acids, or proteins. The term “isolated” encompasses a bacterium or other entity or substance that has been separated from at least some of the components with which it was associated when initially produced (whether in nature or in an experimental setting). In various embodiments, an isolated bacterium or other entity or substance is produced, prepared, purified, and/or manufactured by the hand of man. For example, isolated bacteria may be separated from at least about 10%, about 20%, about 30%, about 40%, about 50%, about 60%, about 70%, about 80%, about 90%, about 95%, about 96%, about 97%, about 98%, about 99% or more of the other components with which they were initially associated (e.g., by weight, such as dry weight). In some embodiments, isolated bacteria are more than about 80%, about 85%, about 90%, about 91%, about 92%, about 93%, about 94%, about 95%, about 96%, about 97%, about 98%, about 99%, or more than about 99% pure (e.g., by weight, such as dry weight). As used herein, a substance is “pure” if it is substantially free of other components. As used herein, an “isolated” or “purified” compound (such as a nucleic acid molecule, polynucleotide, polypeptide, or protein), is substantially free of other cellular material, or culture medium when produced by recombinant techniques, or chemical precursors or other chemicals when chemically synthesized. Purified compounds are at least 60% by weight (dry weight) the compound of interest. Preferably, the preparation is at least 75%, more preferably at least 90%, and most preferably at least 99%, by weight the compound of interest. For example, a purified compound is one that is at least 90%, 91%, 92%, 93%, 94%, 95%, 98%, 99%, or 100% (w/w) of the desired compound by weight. Purity may be measured by any appropriate standard method, for example, by column chromatography, thin layer chromatography, or high-performance liquid chromatography (HPLC) analysis. In certain embodiments, a purified or isolated polynucleotide (ribonucleic acid (RNA) or deoxyribonucleic acid (DNA)) is free of the genes or sequences that flank it in its naturally-occurring state. In various embodiments, purified also defines a degree of sterility that is safe for administration to a human subject, e.g., lacking infectious or toxic agents.

In various embodiments, inactivated, non-viable, or dead Bacillus coagulans bacteria or particles comprising such bacteria comprise inactivated, non-viable, or dead Bacillus coagulans vegetative bacteria. In some embodiments, inactivated, non-viable, or dead Bacillus coagulans bacteria or particles comprising such bacteria comprise inactivated, non-viable, or dead Bacillus coagulans spores. In certain embodiments, inactivated non-viable, or dead Bacillus coagulans bacteria or particles comprising such bacteria comprise inactivated, non-viable, or dead Bacillus coagulans vegetative bacteria and inactivated, non-viable, or dead Bacillus coagulans spores.

In various embodiments, inactivated, non-viable, or dead Bacillus coagulans bacteria are heat and/or pressure inactivated or killed Bacillus coagulans. In certain embodiments, the inactivated, non-viable, or dead Bacillus coagulans bacteria are acid-inactivated or in the form of acidified particles. In some embodiments, dead Bacillus coagulans vegetative cells are at least partially intact, e.g., at least about or about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the surface area (e.g., the surface area compared to a living cell) of the cell walls of the dead Bacillus coagulans vegetative cells is intact. In certain embodiments, at least about or about 1%, 2%, 3%, 4%, 5%6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the dead Bacillus coagulans vegetative cells are at least partially intact, e.g., at least about or about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the surface area of the cell walls of the dead Bacillus coagulans vegetative cells is intact. In various embodiments, the dead Bacillus coagulans vegetative cells are intact.

In some embodiments, inactivated, non-viable, or dead Bacillus coagulans bacteria (e.g., vegetative bacteria and/or spores) have at least about 80%, 85%, 90%, 95%, 96%, 97%, 98% 99%, or 100% of the mass (e.g. dry weight or weight in solution) of a corresponding number of viable Bacillus coagulans bacteria. In certain embodiments, the inactivated, non-viable, or dead Bacillus coagulans bacteria (e.g., vegetative bacteria and/or spores) have at least about 80%, 85%, 90%, 95%, 96%, 97%, 98%, 99%, or 100% of the mass (e.g., dry weight or weight in solution) of the Bacillus coagulans bacteria from which inactivated, non-viable, or dead Bacillus coagulans bacteria were produced (e.g., at the time the process of killing or inactivation began or occurred).

In some embodiments, the dead Bacillus coagulans bacteria comprise fragments or components of dead Bacillus coagulans bacteria. In certain embodiments, the fragments or components comprise fragments or components of Bacillus coagulans bacterial cell walls. In various embodiments, the fragments or components are in the form of (e.g. are aggregated into) particles. In some embodiments, such particles include particles of various sizes, e.g., comprising an approximate maximum width or diameter of about 1-5 μm, 1-10 μm, 5-50 μm, 5-100 μm, 5-25 μm, 10-100 μm, or 1-1000 μm (e.g., about 1 μm, about 2.5 μm, about 5 μm, about 7.5 μm, about 10 μm, about 15 μm, about 20 μm, about 25 μm, about 30 μm, about 35 μm, about 40 μm, about 45 μm, about 50 μm, about 75 μm, about 100 μm, about 150 μm, about 200 μm, about 250 μm, about 300 μm, about 350 μm, about 400 μm, about 450 μm, about 500 μm, about 600 μm, about 700 μm, about 800 μm, about 900 μm, about 1000 μm, 10-1000 μm, 10-100 μm, 10-500 μm, 50-100 μm, 50-200 μm, 50-300 μm, 50-400 μm, 50-500 μm, 100-250 μm, 100-500 μm, 150450 μm, 150-300 μm, 250-500 μm, 500-750 μm, or 500-1000 μm); comprising an average diameter of about 1-5 μm, 1-10 μm, 5-50 μm, 5-100 μm 5-25 μm, 10-100 μm, or 1-1000 μm (e.g., about 1 μm, about 2.5 μm about 5 μm, about 7.5 μm, about 10 μm, about 15 μm, about 20 μm, about 25 μm, about 30 μm, about 35 μm, about 40 μm, about 45 μm, about 50 μm, about 75 μm, about 100 μm, about 150 μm, about 200 μm about 250 μm, about 300 μm, about 350 μm, about 400 μm, about 450 μm, about 500 μm, about 600 μm, about 700 μm, about 800 μm, about 900 μm, about 1000 μm, 10-1000 μm, 10-100 μm, 10-500 μm, 50-100 μm, 50-200 μm, 50-300 μm, 50-400 μm, 50-500 μm, 100-250 μm, 100-500 μm, 150450 μm, 150-300 μm, 250-500 μm, 500-750 μm, or 500-1000 μm; and/or having a total volume of about 10-10000 mm³ (e.g., about 10 mm³, about 25 mm³, about 50 mm³, about 75 mm³, about 100 mm³, about 150 mm³, about 200 mm³, about 250 mm³, about 300 mm³, about 350 mm³, about 400 mm³, about 450 mm³, about 500 mm³, about 600 mm³, about 700 mm³, about 800 mm³, about 900 mm³, about 1000 mm³, about 2500 mm³, about 7000 mm³, about 7500 mm³, about 10000 mm³, 100-10000 mm³, 100-1000 mm³, 100-5000 mm³, 500-1000 mm³, 500-2000 mm³, 500-3000 mm³, 5004000 mm³, 500-5000 mm³, 100-250 mm³, 100-500 mm³, 150-450 mm³, 150-300 mm³, 250-500 mm³, 500-750 mm³, 500-1000 mm³, 1000-2500 mm³, 1000-5000 mm³, 1500-4500 mm³, 1500-3000 mm³, 2500-5000 mm³, 5000-7500 mm³, or 5000-10000 mm³.

In various embodiments, a composition or method provided herein comprises a component of a dead Bacillus coagulans cell wall (e.g., lipoteichoic acid and/or peptidoglycan). In certain embodiments, the component is purified.

In some embodiments, killing a Bacillus coagulans bacterium comprises exposing the Bacillus coagulans bacterium to heat, e.g., under wet (e.g., in an aqueous solution or in the presence of steam) or dry conditions. In certain embodiments, killing the Bacillus coagulans bacterium comprises exposing the Bacillus coagulans bacterium to pressure. In various embodiments, killing the Bacillus coagulans bacterium comprises exposing the Bacillus coagulans bacterium to both heat and pressure. In certain embodiments, killing the Bacillus coagulans bacterium comprises applying pressure to the Bacillus coagulans bacterium with a French press.

In some embodiments, vegetative Bacillus coagulans bacterial cells are killed by one or more methods, e.g., repeated freeze-thaw cycles (e.g., freezing than thawing at least about 2, 3, 4, 5, 6, 7, 8, 9, or 10 times). In certain embodiments, Bacillus coagulans bacteria (e.g., vegetative bacteria and/or spores) are killed by bead milling. In various embodiments, bead milling comprises vortexing the bacteria in the presence of beads. In some embodiments, the beads have a diameter of about 50 μm, 100 μm, 150 μm, 200 μm, 250 μm, 300 μm, 350 μm, 400 μm, 450 μm, 500 μm, 20-250 μm, 100-300 μm, 50-500 μm, 50-750 μm, 500-1000 μm, or 750-1000 μm. In certain embodiments, the beads are low-protein-binding beads. In various embodiments, the beads comprise zirconium. In some embodiments, the beads are zirconium beads. In certain embodiments, killing the Bacillus coagulans bacteria comprises freeze-thaw cycles and bead milling. In various embodiments, killing the Bacillus coagulans does not comprise bead milling. In some embodiments, killing the Bacillus coagulans bacteria comprises drying, e.g. lyophilizing, vegetative bacteria. In certain embodiments, killing the Bacillus coagulans bacteria comprises lyophilizing vegetative bacteria and then milling the lyophilized bacteria (e.g., with beads). In some embodiments, killing the Bacillus coagulans bacteria comprises sonication.

In certain embodiments, Bacillus coagulans bacteria are cultured, e.g., in a fermentor, prior to being inactivated (e.g., killed). In various embodiments, Bacillus coagulans bacteria are centrifuged (e.g., to form a pellet of Bacillus coagulans bacteria) from culture media (e.g., from a fermentor or flask).

In various embodiments, the Bacillus coagulans bacteria are killed as part of the normal manufacturing process of an edible composition. For example, a pasteurization technique that kills all or substantially all (such as about or at least 90%, 91%, 92%, 93%, 94%, 95%, 96%, 97%, 98%, 99%, 99.1%, 99.2%, 99.3%, 99.4%, 99.5%, 99.6%, 99.7%, 99.8% or 99.9%) Bacillus coagulans bacteria (e.g., vegetative cells and/or spores) in a composition may be used. In some embodiments, foods that are pre-cooked during the manufacturing process (such as baked compositions, meat products such as hamburger patties, pre-cooked frozen products, etc.) are cooked at a temperature and/or pressure that kills all or substantially all (such as about or at least 90%, 91%, 92%, 93% 94%, 95%, 96%, 97%, 98%, 99%, 99.1%, 99.2%, 99.3%, 99.4%, 99.5%, 99.6%, 99.7%, 99.8%, or 99.9%) Bacillus coagulans vegetative cells and/or spores in a composition. A non-limiting example with respect to beverage compositions includes beverage compositions that are heated (e.g., boiled and/or steeped) for an amount of time that is sufficient to kill all or substantially all (such as about or at least 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9%) Bacillus coagulans vegetative cells and/or spores in the composition. Non-limiting examples of beverage compositions include hot beverage compositions such as aleberry, anijsmelk, apple cider, asiático, atoly, bajigur, bandrek, blackberry demitasse, blue blazers, bouillon, butter tea, caudle, coffee, hot egg drinks, espresso, hot ginger cordials, greyana rakiya, grog, tea, hot buttered rum, hot chocolate, hot toddies, Irish coffee, hot lemonade, malted milk, mate cocido, mulled wine, posset, postum, sake, salep, sassafras tea, smoking bishop, hot sodas, spiced punch, and wedang jahe.

In some embodiments, the genomes of the inactivated, non-viable, or dead Bacillus coagulans bacteria are intact, or partially intact so as to be identifiable as B. coagulans. In certain embodiments, the inactivated, non-viable, or dead Bacillus coagulans bacteria can be identified as containing Bacillus coagulans genomic DNA, e.g., by sequencing, polymerase chain reaction, microarray analysis, and/or probes. In various embodiments, at least 1 or more 10, 20, 30, 40, 50, 60, 70, 80, 90, 100, 150, 200, 250, 300, 350, 400, 450, 500, 750, 1000, 1500, 2000, 2500, or 3000 kilobase fragment or portion of the Bacillus coagulans genome is present within inactivated, non-viable, or dead Bacillus coagulans vegetative bacteria and/or spores.

In certain embodiments, a composition provided herein is present on the exterior surface of an edible composition. For example the composition may be present as a coating on the exterior surface of the edible composition. In some embodiments, the composition completely surrounds the edible composition. In various embodiments, the edible composition comprises a food composition or a supplement composition. In some embodiments, the edible composition comprises a food composition. In certain embodiments, an edible composition may be edible for and/or fed to a human or a non-human animal (e.g., a reptile, amphibian, bird, or mammal) such as a primate (e.g., an ape, monkey, gorilla, orangutan, chimpanzee, or human) or other animal (e.g., a parrot, chicken, goose, duck, dog, cat, rabbit, pig, or horse, or a ruminant such as a cow, sheep, goat, buffalo, yak, deer, elk, giraffe, or camel). In some embodiments, the animal is a pseudoruminant, such as a hippopotamus. Ruminants have four-chambered stomachs whereas pseudoruminants have three-chambered stomachs. In some embodiments, the animal is a performance animal such as a military dog, a police dog, a race dog, a show dog, a military horse, a police horse, a race horse, a polo horse, or a show horse.

In some embodiments, the inactivated, non-viable, or dead Bacillus coagulans bacteria are in the form of particles of inactivated, non-viable, or dead Bacillus coagulans bacteria. In certain embodiments, a composition provided herein is in the form of or comprises a particle or particles (e.g., at least about 1, 5, 10, 50, 100, 500, 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, or 1×10²⁰ particles, e.g., total or per gram). In various embodiments, at least 80%, 85%, 90%, 95%, 96%, 97% 98%, 99%, 99.1%, 99.2%, 99.3%, 99.4%, 99.5%, 99.6%, 99.7%, 99.8%, 99.9%, or 100% of the particles are 25 mesh (i.e., small enough to pass through a sieve with a nominal opening of 707 μm), 30 mesh (i.e., small enough to pass through a sieve with a nominal opening of 595 μm), 35 mesh (i.e., small enough to pass through a sieve with a nominal opening of 500 μm), 40 mesh (i.e., small enough to pass through a sieve with a nominal opening of 420 μm), 45 mesh (i.e., small enough to pass through a sieve with a nominal opening of 354 μm), or 50 mesh (i.e., small enough to pass through a sieve with a nominal opening of 297 μm) and at least 70%, 75%, 80%, 85%, 90%, 95%, 96%, 97%, 98%, 99%, 99.1%, 99.2%, 99.3%, 99.4%, 99.5%, 99.6%, 99.7%, 99.8%, 99.9%, or 100%% of the particles are 70 mesh (i.e., small enough to pass through a sieve with a nominal opening of 210 μm), 80 mesh (i.e., small enough to pass through a sieve with a nominal opening of 177 μm), or 100 mesh (i.e., small enough to pass through a sieve with a nominal opening of 149 μm). In some embodiments, at least 80%, 85%, 90%, 95%, 96%, 97%, 98%, 99%, 99.1%, 99.2%, 99.3%, 99.4%, 99.5%, 99.6%, 99.7%, 99.8%, 99.9%, or 100% of the particles are have at least one dimension that is less than about 707 μm, 595 μm, 500 μm, 420 μm, 354 μm, or 297 μm and at least 70%, 75%, 80%, 85%, 90%, 95%, 96%, 97%, 98%, 99%, 99.1%, 99.2%, 99.3%, 99.4%, 99.5%, 99.6%, 99.7%, 99.8%, 99.9%, or 100% of the particles have at least one dimension that is less than about 210 μm 177 μm, or 149 μm. In certain embodiments, at least 80%, 85%, 90%, 95%, 96%, 97%, 98%, 99%, 99.1%, 99.2%, 99.3%, 99.4%, 99.5%, 99.6%, 99.7%, 99.8%, 99.9%, or 100% of the particles are have at least two dimensions that are less than about 707 μm, 595 μm, 500 μm, 420 μm, 354 μm, or 297 μm and at least 70%, 75%, 80%, 85%, 90%, 95%, 96%, 97%, 98%, 99%, 99.1%, 99.2%, 99.3%, 99.4%, 99.5%, 99.6%, 99.7%, 99.8%, 99.9%, or 100% of the particles have at least two dimensions that are less than about 210 μm, 177 μm, or 149 μm. In various embodiments, at least 80%, 85%, 90%, 95%, 96%, 97%, 98%, 99%, 99.1%, 99.2%, 99.3%, 99.4%, 99.5%, 99.6%, 99.7%, 99.8%, 99.9%, or 100% of the particles have no dimension that is greater than about 707 μm, 595 μm, 500 μm, 420 μm, 354 μm, or 297 μm and at least 70%, 75%, 80%, 85%, 90%, 95%, 96%, 97%, 98%, 99%, 99.1%, 99.2%, 99.3%, 99.4%, 99.5%, 99.6%, 99.7%, 99.8%, 99.9%, or 100% of the particles have no dimension that is greater than about 210 μm, 177 μm, or 149 μm.

In certain embodiments, the particles are present within a composition (such as an edible composition). In various embodiments, the particle are present on the exterior surface of composition (such as an edible composition). In some embodiments, particles are present within and on the exterior surface of composition (such as an edible composition).

The term “particle,” as used herein, refers to a discrete body. Particles may be amorphous, or may take a variety of shapes, including round, oblong, square, etc. Non-limiting examples of particles include crystals, grains, beads, amorphous bodies, and spheres. Certain embodiments of the present subject matter include particles of various sizes, e.g., comprising an approximate maximum width or diameter of about 1-5 μm, 1-10 μm, 5-50 μm, 5-100 μm 5-25 μm, 10-100 μm, or 1-1000 μm (e.g., about 1 μm, about 2.5 μm, about 5 μm, about 7.5 μm about 10 μm, about 15 μm, about 20 μm, about 25 μm, about 30 μm, about 35 μm, about 40 μm, about 45 μm, about 50 μm, about 75 μm, about 100 μm, about 150 μm, about 200 μm, about 250 μm, about 300 μm, about 350 μm, about 400 μm, about 450 μm, about 500 μm, about 600 μm, about 700 μm, about 800 μm, about 900 μm, about 1000 μm, 10-1000 μm, 10-100 μm, 10-500 μm, 50-100 μm, 50-200 μm, 50-300 μm, 50400 μm, 50-500 μm, 100-250 μm 100-500 μm, 150-450 μm, 150-300 μm, 250-500 μm 500-750 μm, or 500-1000 μm); comprising an average diameter of about 1-5 μm, 1-10 μm, 5-50 μm, 5-100 μm, 5-25 μm, 10-100 μm, or 1-1000 μm (e.g., about 1 μm, about 2.5 μm, about 5 μm, about 7.5 μm, about 10 μm about 15 μm, about 20 μm, about 25 μm, about 30 μm, about 35 μm, about 40 μm, about 45 μm, about 50 μm, about 75 μm, about 100 μm about 150 μm, about 200 μm, about 250 μm, about 300 μm, about 350 μm, about 400 μm, about 450 μm, about 500 μm, about 600 μm, about 700 μm, about 800 μm, about 900 μm, about 1000 μm, 10-1000 μm, 10-100 μm 10-500 μm, 50-100 μm, 50-200 μm, 50-300 μm, 50400 μm, 50-500 μm, 100-250 μm, 100-500 μm, 150-450 μm 150-300 μm 250-500 μm 500-750 μm, or 500-1000 μm; and/or having a total volume of about 10-10000 mm³ (e.g., about 10 mm³, about 25 mm³, about 50 mm³, about 75 mm³, about 100 mm³, about 150 mm³, about 200 mm³, about 250 mm³, about 300 mm³, about 350 mm³, about 400 mm³, about 450 mm³, about 500 mm³, about 600 mm³, about 700 mm³, about 800 mm³, about 900 mm³, about 1000 mm³, about 2500 mm³, about 7000 mm³, about 7500 mm³, about 10000 mm³, 100-10000 mm³, 100-1000 mm³, 100-5000 mm³, 500-1000 mm³, 500-2000 mm³, 500-3000 mm³, 500-4000 mm³, 500-5000 mm³, 100-250 mm³, 100-500 mm³, 150450 mm³, 150-300 mm³, 250-500 mm³, 500-750 mm³, 500-1000 mm³, 1000-2500 mm³, 1000-5000 mm³, 1500-4500 mm³, 1500-3000 mm³, 2500-5000 mm³, 5000-7500 mm³, or 5000-10000 mm³. In some embodiments, a particle comprises at least about 1, 5, 10, 50, 100, 500, 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, or 1×10²⁰ inactivated, non-viable, or dead Bacillus coagulans vegetative bacteria and/or spores, or about 1×10³ to about 1×10⁵, about 1×10³ to about 1×10⁶, about 1×10³ to about 1×10⁷, about 1×10³ to about 1×10⁸, about 1×10³ to about 1×10⁹, about 1×10³ to about 1×10¹⁰, about 1×10³ to about 1×10¹¹, about 1×10³ to about 1×10¹², about 1×10³ to about 1×10¹³, about 1×10³ to about 1×10¹⁴, 1×10⁶ to about 1×10⁵, about 1×10⁶ to about 1×10⁶, or about 1×10⁶ to about 1×10⁷, about 1×10⁶ to about 1×10⁸, about 1×10⁶ to about 1×10⁹, about 1×10⁶ to about 1×10¹⁰, about 1×10⁶ to about 1×10¹¹, about 1×10⁶ to about 1×10¹², about 1×10⁶ to about 1×10¹³, about 1×10⁶ to about 1×10¹⁴, 1×10³-1×10²⁰, 1×10³-1×10⁹, 1×10³-1×10⁶, 1×10⁶-1×10⁹, 1×10⁶-1×10²⁰, 1×10⁶-1×10¹⁵, 1×10⁶-1×10¹², 1×10⁸-1×10¹⁷, 1×10¹⁰-1×10²⁰, 1×10³-1×10¹⁰, 1×10⁵-1×10¹⁰, 1×10⁵-1×10¹⁵, 1×10¹⁰-1×10¹⁵, 1×10¹⁴-1×10¹⁶, or about at least, or less than 5, 10, 50, 100, 500, 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, or 1×10²⁰ inactivated, non-viable, or dead Bacillus coagulans vegetative bacteria and/or spores.

In certain embodiments, a composition comprises at least about 1, 5, 10, 50, 100, 500, 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, or 1×10²⁰ inactivated, non-viable, or dead Bacillus coagulans vegetative bacteria and/or spores, or about 1×10³ to about 1×10⁵, about 1×10³ to about 1×10⁶, about 1×10³ to about 1×10⁷, about 1×10³ to about 1×10⁸, about 1×10³ to about 1×10⁹, about 1×10³ to about 1×10¹⁰, about 1×10³ to about 1×10¹¹, about 1×10³ to about 1×10¹², about 1×10³ to about 1×10¹³, about 1×10³ to about 1×10¹⁴, 1×10⁶ to about 1×10⁵, about 1×10⁶ to about 1×10⁶, or about 1×10⁶ to about 1×10⁷, about 1×10⁶ to about 1×10⁸, about 1×10⁶ to about 1×10⁹, about 1×10⁶ to about 1×10¹⁰, about 1×10⁶ to about 1×10¹¹, about 1×10⁶ to about 1×10¹², about 1×10⁶ to about 1×10¹³, about 1×10⁶ to about 1×10¹⁴, 1×10³-1×10²⁰, 1×10³-1×10⁹, 1×10³-1×10⁶, 1×10⁶-1×10⁹, 1×10⁶-1×10²⁰, 1×10⁶-1×10¹⁵, 1×10⁶-1×10¹², 1×10⁸-1×10¹⁷, 1×10¹⁰-1×10²⁰, 1×10³-1×10¹⁰, 1×10⁵-1×10¹⁰, 1×10⁵-1×10¹⁵, 1×10¹⁰-1×10¹⁵, 1×10¹⁴-1×10¹⁶, or about at least, or less than 5, 10, 50, 100, 500, 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, or 1×10²⁰ inactivated, non-viable, or dead Bacillus coagulans vegetative bacteria and/or spores. In various embodiments, the number of inactivated, non-viable, or dead Bacillus coagulans vegetative bacteria and/or spores in a composition is about 1×10⁵-1×10⁷ per gram of about 1×10⁵, 1.5×10⁵, 2×10⁵, 2.5×10⁵, 3×10⁵, 3.5×10⁴, 4×10⁵, 4.5×10⁵, 5×10⁵, 5.5×10⁵, 6×10⁵, 6.5×10⁵, 7×10⁵, 7.5×10⁵, 8×10⁵, 8.5×10⁵, 9×10⁵, 9.5×10⁵, 1×10⁶, 1.5×10⁶, 2×10⁶, 2.5×10⁶, 3×106, 3.5×10⁶, 4×10⁶, 4.5×10⁶, 5×10⁶, 5.5×10⁶, 6×10⁶, 6.5×10⁶, 7×10⁶, 7.5×10⁶, 8×10⁶, 8.5×10⁶, 9×10⁶, 9.5×10⁶, or 1×10⁷, inactivated, non-viable, or dead Bacillus coagulans vegetative bacteria and/or spores per gram.

In various embodiments, the inactivated, non-viable, or dead Bacillus coagulans bacteria are treated to reduce the clumping thereof. In some embodiments, particles of inactivated, non-viable, or dead Bacillus coagulans bacteria are treated to reduce the clumping thereof. In certain embodiments, treating the inactivated, non-viable, or dead bacteria comprises passing the inactivated, non-viable, or dead Bacillus coagulans bacteria through a sieve or filter. In various embodiments, treating the inactivated, non-viable, or dead Bacillus coagulans bacteria comprises combining the inactivated, non-viable, or dead Bacillus coagulans bacteria with a surfactant or emulsifier. In some embodiments, treating the inactivated, non-viable, or dead Bacillus coagulans bacteria comprises combining the inactivated, non-viable, or dead Bacillus coagulans bacteria with polysorbate 20 and/or polysorbate 80. In certain embodiments, treating the inactivated, non-viable, or dead Bacillus coagulans bacteria comprises combining the inactivated, non-viable, or dead Bacillus coagulans bacteria with maltodextrin, inulin, inositol, trehalose, micro-crystalline cellulose (MCC), calcium lactate, magnesium stearate, fructo-oligosaccharide (FOS), or gluco-oligosaccharide (GOS).

In various embodiments, inactivated, non-viable, or dead Bacillus coagulans bacteria (e.g., alone or as part of a coating composition) are or have been applied to an external surface (e.g., as a coating on one or more surfaces, e.g. the top surface or the entire surface) by a physical process. Non-limiting examples of physical processes include atomization coating, spray dry coating, spinning disk coating, extrusion coating, fluidized bed coating, pan coating, dripping, emulsion coating, suspension coating, and centrifugal extrusion coating.

The form of administration of the inactivated probiotic in the method of the invention is not critical, as long as an effective amount is administered to reduce the stress and/or anxiety of the subject. As used herein, “effective” when referring to an amount of an inactivated, non-viable, or dead Bacillus coagulans bacterium (or particles comprising inactivated, non-viable, or dead Bacillus coagulans bacteria) refers to the quantity of the inactivated, non-viable, or dead Bacillus coagulans bacteria (or particle) that is sufficient to yield a desired response, in this case, the stress and/or anxiety of the subject, without undue adverse side effects (such as toxicity, irritation, or allergic response) commensurate with a reasonable benefit/risk ratio when used in the manner of this disclosure. The stress and/or anxiety reduction can be self-described and/or measured by a health professional, e.g. reduction in cortisol levels as compared to cortisol levels of the subject when stressed or anxious, decreased blood pressure, normal heart rate, etc.

In some embodiments, the inactivated, non-viable, or dead Bacillus coagulans bacteria (or particles comprising inactivated, non-viable, or dead Bacillus coagulans bacteria) are administered to a subject via tablets, pills, encapsulations, caplets, gel caps, capsules, oil drops, or sachets. In certain embodiments, the inactivated, non-viable, or dead Bacillus coagulans bacteria or particles, are encapsulated in a sugar, fat, or polysaccharide. In various embodiments, inactivated, non-viable, or dead Bacillus coagulans bacteria or particles, are added to a food or drink product and consumed. In some embodiments, the food or drink product is a nutritional product for children such as a follow-on formula, growing up milk, beverage, milk, yogurt, fruit juice, fruit-based drink, chewable tablet, cookie, cracker, or a milk powder. In certain embodiments, the product is an infant nutritional product, such as an infant formula or a human milk fortifier.

In some embodiments, the edible composition comprises a hard sweet, fudge, toffee, liquorice, chocolate, jelly candy, marshmallow, and marzipan. In various embodiments, the edible composition comprises chocolate. For example, the edible composition may include a candy bar comprising chocolate and at least one other ingredient.

Various implementations provide edible compositions with inactivated, non-viable, or dead Bacillus coagulans bacteria or particles the exterior surface thereof. Alternatively or in addition, inactivated, non-viable, or dead Bacillus coagulans bacteria may be present within the edible composition. For example, inactivated, non-viable, or dead Bacillus coagulans bacteria or particles may be on the exterior surface of and/or inside the edible composition.

Also provided are sports nutrition compositions for relieving or ameliorating stress or stress-related injury, e.g., muscle pains, neck pain, back pain etc. In some embodiments, the sports nutrition composition comprises at least about 10%, 20%, 30%, 40%, 50% or 60% protein by dry weight. A non-limiting example of sports nutrition compositions contain at least about 5, 10, 15, 20, 25, 30, 36, 40, 45, 50, 55, 60, 65, 70, 75, 80, 85, 90, 95, or 100 grams of carbohydrates and/or at least about 5, 10, 15, 20, 25, 30, 36, 40, 45, 50, 55, 60, 65, 70, 75, 80, 85, 90, 95, or 100 grams of protein. A non-limiting example of a sports nutrition composition includes protein powder for use in, e.g., a shake, pre-workout, or post-workout beverage. In some embodiments, the sports nutrition composition includes a vitamin or a mineral. Non-limiting examples of sports nutrition compositions include sports nutrition bars such as an energy bars, protein bars, endurance bars, meal replacement bars, pre-workout bars, and post-workout bars. Such sports nutrition bars may comprise, e.g., dried or dry grains (e.g., barley, oats, rice, rye, spelt, teff, triticale, wheat, sorghum, millet, maize, and/or fonio), nuts (e.g., almonds, brazil nuts, candlenuts, cashews, hazelnuts, macadamia nuts, chestnuts, pecans, peanuts, mongongo, pine nuts, pistachios, walnuts, and/or yeheb nuts), dried fruit, honey, animal protein, whey protein, vegetable protein, vitamins, minerals, sugars, fiber, and/or starches.

In some embodiments, the edible composition is a beverage. Non-limiting examples of beverages include tea, green tea, black tea, oolong tea, yellow tea, white tea, herbal tea, rosehip tea, chamomile tea, jiaogulan tea, ginger tea, peppermint tea, fruit tea, jasmine tea, hibiscus tea, lemongrass tea, ginseng tea, rooibos tea, coffee, juice, apple juice, coconut water, cranberry juice, grape juice, grapefruit juice, kiwi fruit juice, lemonade, lemon juice, limeade, lime juice, limonana, melon juice, mora must, orange juice, papaya juice, pineapple juice, pomegranate juice, prune juice, strawberry juice, tomato juice, beet juice, carrot juice, celery juice, cucumber juice, dandelion-green juice, spinach juice, turnip juice, soda, orange soda, cola soda, root beer soda, cream soda, water, mineral water, seltzer water, or tonic water.

In certain embodiments, the edible composition is an alcoholic beverage. In various embodiments, the alcoholic beverage is beer, wine, or a spirit. In some embodiments, the alcoholic beverage is at least about 1, 2, 3, 4, 5, 10, 15, 20, 25, 30, 35, 40, 45, 50, 55, 60, 65, 70, 75, 80, 85, 90, or 95% ethanol by volume. In certain embodiments, the level of ethanol is effective to kill all vegetative Bacillus coagulans vegetative bacteria and/or spores that are added to it. Non-limiting examples of alcoholic beverages include beer, ale, barley wine, whiskey, shōchu, vodka, brem, tongba, boza, brem, huangjiu, choujiu, ruou gao, sake, sonti, makgeolli, chungju, tuak, thwon, kvass, burukutu, pito, merisa, bilibili, maotai, gaoliang, weizenkorn, sojum, horilka, cider, applejack, calvados, jabukovac̆a, kajsijevac̆a, kaisieva rakia, pálinka, feni, majmunovac̆a, chuoi hot, caim, urgwagwa, mbege, kasikisi, arrack, kirsch, ginger ale, ginger beer, ginger wine, gouqi jiu, red wine, white rine, rosé, lozovc̆a, vinjak, brandy, cognac, vermouth, armagnac, branntwein, pisco, rakia, singani, arak, törkölypálinka, gin, genever, borovic̆ka, oghi, poiré, pear cider, plum wine, viljamovka, pear brandy, eau-de-vie, pálinka, krushova rakia, s̆ljivovica, slivovitz, ţuică, umeshu, pálinka, slivova rakia, dunjevc̆a, pomace wine, grappa, marc, orujo, tequila, mezcal, raicilla, pulque, cauim, chicha, kasiri, nihamanchi, mead, sambuca, and absinthe.

In various embodiments, the edible composition is a fermented food or beverage. Non-limiting examples of fermented foods and beverages include amasi, amazake, appam, atchara, ayran, bagoong, bagoong monamon, bagoong terong, bánh cu\hat{ó}n, beer, bland, boza, leavened bread, brem, burong mangga, buttermilk, calpis, chass, cheeses such as shanklish, cheonggukjang, chakuli pitha, fermented cod liver oil, crème fraiche, curtido, dhokla, doenjang, doogh, dosa, doubanjiang, douchi, enduri pitha, fermended bean curd, fermented bean paste, fermented fish, fermented milk products, filmjölk, ganjang, garri, gejang, gochujang, gundruk, hákarl, hongeohoc, idli, igunaq, ingera, iru, jeotgal, jogijeot, kapusta kiszona duszona, katsuobushi, kaymak, kefir, kenkey, khanom chin, kimchi, kiviak, kombucha, kumis, kusaya, kuzhi paniyaram, kvass, lassi, leben, lufu, mageu, massa de pimentão, meigan cai, miso, mixian, mohnyin tjin, murri, mursik, myeolchijeot, nata de coco, nattō, nem chua, ngapi, ogi, ogiri, oncom, palappam, pesaha appam, peuyeum, pickles, podpiwek, poi, pon ye gyi, pulque, puto, rakfisk, ruo̧u, n\hat{é}p, ryazhenka, saeujeot, salami, sauerkraut, symbiotic cultures of bacteria and yeast, Şalgam, shiokara, fermented shrimp paste, sinki, skyr, smântână, smetana, som moo, sour cabbage, sour cream, soured milk, sowans, soy sauce, ssamjang, stinky tofu, strained yogurt, suan cai, sumbala, surströmming, tapai, tarhana, tempeh, tesgiiino, tianmianjiang, tianmianjiang, tibicos, tsukemono, tuong, tung tsai, villi, vinegar, wine, white sugar sponge cake, Worcestershire sauce, yakult, fermented yellow soybean paste, yogurt, zha cai, and Z̆inc̆ica.

In some embodiments, the edible composition comprises a soup. In certain embodiments, the edible composition comprises a grain. In various embodiments, the edible composition comprises a grain-based composition. In some embodiments, the grain-based composition comprises pasta, oatmeal, grits, or cereal. Non-limiting examples of pasta include tubular pasta, straight round rod pasta, ribbon pasta, micro pasta, stuffed pasta, irregular-shaped pasta, spaghetti (solid, thin cylinders), macaroni (tubes or hollow cylinders), fusilli (spiral-shaped), lasagna (sheets), tagliatelle (flat ribbons), vermicelli (thin spaghetti), and ravioli (filled pasta), penne (cylinder-shaped pasta), rotini (corkscrew-shaped pasta), rigatoni (tube-shaped pasta), noodles, and spätzle. In certain embodiments, the pasta is dried. In various embodiments, the pasta is fresh. In some embodiments, the pasta comprises egg pasta. In some embodiments, the pasta does not include egg.

In certain embodiments, the edible composition comprises a baked composition. In various embodiments, the baked composition comprises a bread, a cake, a muffin, a pie, a tart, a pastry, a food bar, a granola bar, a quiche, a cookie, a pizza, a baked corn chip, a baked tortilla chip, a baked potato chip, a baked cracker, and baked treats for companion animals.

In some embodiments, the composition comprises a dairy composition. In certain embodiments, the composition comprises a milk-like composition.

In various embodiments, the inactivated, non-viable, or dead Bacillus coagulans bacteria or particles are administered orally. In some embodiments, the inactivated, non-viable, or dead Bacillus coagulans bacteria or particles are administered nasally, topically (e.g., to a mucus membrane and/or to the skin), intraperitoneally, or intravenously.

In certain embodiments, a composition is formulated as a tablet, capsule, lozenge, aqueous solution, syrup, suspension, emulsion, powder, gel, lotion, or cream.

In some embodiments, the composition is a supplement composition. A supplement composition is a composition comprising an added dietary supplement. As used herein, the term “dietary supplement” is defined as in the United States Dietary Supplement Health and Education Act of 1994 (DSHEA). In various embodiments, a dietary supplement is a product (other than tobacco) intended to supplement the diet that bears or contains one or more of the following dietary ingredients: (A) a vitamin; (B) a mineral; (C) an herb or other botanical; (D) an amino acid; (E) a dietary supplement used by man to supplement the diet by increasing the total dietary intake; or (F) a concentrate, metabolite, constituent, extract, or combination of any ingredient described in (A), (B), (C), (D), or (E).

Various strains of Bacillus coagulans may be used to create inactivated, non-viable, or dead Bacillus coagulans bacteria in compositions, coatings, particles, and edible compositions of the present subject matter. For example, the Bacillus coagulans may include one or more of Bacillus coagulans Hammer strain Accession No. ATCC 31284, a strain derived from Bacillus coagulans Hammer strain Accession No. ATCC 31284, GBI-30 strain (ATCC Designation Number PTA-6086), GBI-20 strain (ATCC Designation Number PTA-6085), or GBI-40 strain (ATCC Designation Number PTA-6087).

The present subject matter provides compositions comprising inactivated, non-viable, or dead Bacillus coagulans bacteria (e.g., particles comprising inactivated, non-viable, or dead Bacillus coagulans bacteria and compositions comprising such particles). In some embodiments, the inactivated, non-viable, or dead Bacillus coagulans bacteria or particles comprising such bacteria may be in the form of a dry mix that is suitable for addition to, e.g., food compositions. In certain embodiments, the dry mix may be between 1% and 50% inactivated, non-viable, or dead Bacillus coagulans bacteria, e.g., about 5%, about 10%, about 15%, about 20%, about 25%, about 35%, about 45%, or about 50% inactivated, non-viable, or dead Bacillus coagulans bacteria by weight (e.g., dry weight). In various embodiments, the dry mix is about 15% inactivated, non-viable, or dead Bacillus coagulans bacteria by weight. For example, about 100 pounds of dry mix may contain about 15 pounds of inactivated, non-viable, or dead Bacillus coagulans bacteria.

The Bacillus coagulans Hammer strains are non-pathogenic and generally regarded as safe for use in human nutrition (i.e., GRAS classification) by the U.S. Federal Drug Administration (FDA) and the U.S. Department of Agriculture (USDA), and by those skilled in the art. Furthermore, the Bacillus coagulans Hammer strains described herein germinate at or below human body temperature, rendering them useful as probiotics. Many Bacillus coagulans strains outside the Hammer group have mostly industrial applications, little or no nutritional benefit, and environmental contaminants that have not been evaluated for safety. Moreover, many other non-Hammer strains of Bacillus coagulans grow optimally at temperatures that exceed human body temperature and, thus, do not germinate efficiently in the human body. Such strains are less or not suitable as probiotics for human consumption.

Unless specifically defined otherwise, all technical and scientific terms used herein shall be taken to have the same meaning as commonly understood by one of ordinary skill in the art (e.g., in cell culture, molecular genetics, and biochemistry).

In the descriptions herein and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B. and C;” “one or more of A, B, and C;” and “A, B, and/or C” are each intended to mean “A alone, B alone, C alone. A and B together, A and C together, B and C together, or A and B and C together.” In addition, use of the term “based on,” herein and in the claims is intended to mean, “based at least in part on,” such that an unrecited feature or element is also permissible.

As used herein, the term “about” in the context of a numerical value or range means±10% of the numerical value or range recited or claimed, unless the context requires a more limited range.

It is understood that where a parameter range is provided, all integers within that range, and tenths thereof, are also provided by the invention. For example, “0.2-5 mg” is a disclosure of 0.2 mg, 0.3 mg, 0.4 mg, 0.5 mg, 0.6 mg etc. up to and including 5.0 mg.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention. Where methods and compositions are disclosed using the transitional term “comprising” it will be understood that corresponding methods and compositions with the transitional term “consisting of” and “consisting essentially of” are also disclosed.

Each embodiment disclosed herein is contemplated as being applicable to each of the other disclosed embodiments. Thus, all combinations of the various elements described herein are within the scope of the invention.

### DETAILED DESCRIPTION

Stress is both a biological and a psychological response. It occurs when a situation is perceived to be challenging or threatening (i.e., meeting a work deadline or facing a speeding car). Stress responses are mediated by the human “stress system,” which may involve the amygdala, hypothalamus, autonomic nervous system, endocrine system, and organs. When the brain perceives a stimulus as a stressor, it sends a distress signal to the hypothalamus, which then activates the sympathetic branch of autonomic nervous system, sending signals to the adrenal glands. The hormone epinephrine is pumped into the bloodstream and acts on the target organs, speeding up the heartbeat and breathing, stiffening the muscles, and causing sweating. The combination of these reactions is known as the “fight-or-flight” response, which enables a rapid reaction to life-threatening (potentially life-threatening) situations and help us fight off threats or flee to safety. The same reaction may occur when an individual perceives a situation or series of situations as being life-threatening (but are not life-threatening). Acute stress is transient, beneficial, and even vital in many cases. When a stressful situation passes, the parasympathetic branch of autonomic nervous system is activated, acting as a “brake” to dampen the stress responses and help to re-establish homeostasis.

However, this “brake” might fail to operate when the body overreacts to some chronic stressors, such as long-term work pressure. When the brain continuously perceives the situation as stressful, always-on “fight-or-flight” responses may put the autonomic nervous system off balance and cause the responsiveness of the stress system to further deteriorate. The cumulative effects of chronic stress often degrade performance in work and relationships with family and friends. Physiologically, long-term activation of adrenal glands can release excess cortisol (a stress hormone) which disrupts various bodily processes and disturbs homeostasis. Elevated cortisol level puts high-stress individuals at an increased risk of numerous health problems, including anxiety, depression, and/or physiological conditions such as heart disease, hypertension, and diabetes.

Proper responsiveness of the stress system is also essential for regulating healthy emotions in social interactions and a sense of well-being, and a number of techniques have been developed for coping with stress and these have been used to help people maintain homeostasis and autonomic balance. For example, listening to relaxing music can reduce cortisol levels and help people recover from periods of stress. Some research suggests that stress recovery is facilitated by exposure to colors and sound stimuli recorded in natural environments. Studies also indicate that meditation practice improves the balance of the autonomic nervous system and that mindfulness practices deliver several positive benefits, including decreased anxiety and increased focus and improved mood. Yoga has been observed to stimulate an underactive parasympathetic nervous system and helps to correct an imbalance of autonomic nervous system. Pharmaceutical anti-depressant or anti-anxiety drugs such as barbiturates, benzodiazepines, buspirone, serotonin reuptake inhibitors (such as Prozac or Celexa), or serotonin-norepinephrine reuptake inhibitors are used for reduction of stress and/or anxiety; however, these medications can be habit-forming and are often associated with adverse side effects such as nausea, drowsiness, dizziness, trouble sleeping, tiredness, loss of appetite, and/or sweating. A reliable, safe, effective, non-pharmaceutical means for reduction of stress and/or anxiety, e.g., as demonstrated by a reduction in cortisol levels in an affected individual, has been elusive. The compositions and method of described herein provide a solution to the long-standing unmet need of stress and/or anxiety management. Ingestion of B. coagulans compositions as described has been shown to improve the body's ability to adapt to challenges including mental or physical stress as well as manifestations thereof including anxiety.

The compositions of the invention are useful to reduce stress and anxiety in individuals suffering from or experiencing symptoms including one or more of the following symptoms or reactions. The individual may experience 1, 2, 3, 4, 5, 10, 20, 25, or more of such symptoms or reactions. The symptoms or reactions may be self-described, self-assessed, or observed/determined by a healthcare worker.

### Behavioral:

Change in activity levels
Decreased efficiency and effectiveness
Difficulty communicating
Increased sense of humor/gallows humor
Irritability, outbursts of anger, frequent arguments
Inability to rest, relax, or let down
Change in eating habits
Change in sleep patterns
Change in job performance
Periods of crying
Increased use of tobacco, alcohol, drugs, sugar or caffeine
Hyper-vigilance about safety or the surrounding environment
Avoidance of activities or places that trigger memories
Accident prone

### Psychological or Emotional:

Feeling heroic, euphoric or invulnerable
Denial
Anxiety or fear
Worry about safety of self or others
Irritability or anger
Restlessness
Sadness, moodiness, grief or depression
Vivid or distressing dreams
Guilt or “survivor guilt”
Feeling overwhelmed, helpless or hopeless
Feeling isolated, lost, lonely or abandoned
Apathy
Over identification with survivors
Feeling misunderstood or unappreciated

### Physical:

Increased heart rate and respirations
Increased blood pressure
Upset stomach, nausea, diarrhea
Increased or decreased appetite which may be accompanied by weight loss or gain
Sweating or chills
Tremors or muscle twitching
Muffled hearing
Tunnel vision
Feeling uncoordinated
Headaches
Sore or aching muscles
Light sensitive vision
Lower back pain
Feeling a “lump in the throat”
Easily startled
Fatigue that does not improve with sleep
Menstrual cycle changes
Change In sexual desire or response
Decreased resistance to colds, flu, infections
Flare up of allergies, asthma, or arthritis
Hair loss

### Cognitive:

Memory problems/forgetfulness
Disorientation
Confusion
Slowness in thinking, analyzing, or comprehending
Difficulty calculating, setting priorities or making decisions
Difficulty Concentrating
Limited attention span
Loss of objectivity
Inability to stop thinking about the disaster or an incident

### Social:

Withdrawing or isolating from people
Difficulty listening
Difficulty sharing ideas
Difficulty engaging in mutual problem solving
Blaming
Criticizing
Intolerance of group process
Difficulty in giving or accepting support or help
Impatient with or disrespectful to others

The present subject matter provides compositions and methods for reducing stress comprising inactivated, non-viable, and/or dead Bacillus coagulans bacteria. Inactivated, non-viable, and/or dead Bacillus coagulans bacteria supports a healthy stress response, as well as mucosal immunity (e.g., the immune system response of the mucous membranes, such as in oral, gut, and lung surfaces). The inactivated, non-viable, and/or dead Bacillus coagulans bacteria support normal inflammatory response to stress, and reduce the body's overall stress response, while maintaining immunity. In various embodiments, the inactivated, non-viable, and/or dead Bacillus coagulans bacteria significantly reduce cortisol by at least 5%, 10%, 20%, 30%, or 40%, e.g. compared to baseline levels (such as levels in blood) or following a physically or emotionally stressful challenge. In some embodiments, the inactivated, non-viable, and/or dead Bacillus coagulans bacteria avoid negative effects of stress, and have an adaptogenic effect (e.g., the body's ability to adapt to challenges including stress improves after administration).

Bacillus coagulans

Bacillus coagulans is a non-pathogenic, Gram positive, spore-forming bacteria that produces L(+) lactic acid (dextrorotatory) under homo-fermentation conditions. It has been isolated from natural sources, such as heat-treated soil samples inoculated into nutrient medium (see e.g., Bergey's Manual of Systemic Bacteriology, Vol. 2, Sneath, P. H. A. et al., eds., Williams & Wilkins, Baltimore, Md., 1986). Isolated Bacillus coagulans strains have served as a source of enzymes including endonucleases (e.g., U.S. Pat. No. 5,200,336); amylase (U.S. Pat. No. 4,980,180); lactase (U.S. Pat. No. 4,323,651) and cyclo-malto-dextrin glucano-transferase (U.S. Pat. No. 5,102,800).

Various Bacillus coagulans bacterial strains which are currently commercially available from the American Type Culture Collection (ATCC, Manassas. Va.) include the following accession numbers: Bacillus coagulans Hammer NRS 727 (ATCC No. 11014); Bacillus coagulans Hammer strain C (ATCC No. 11369); Bacillus coagulans Hammer (ATCC No. 31284); and Bacillus coagulans Hammer NCA 4259 (ATCC No. 15949). Purified Bacillus coagulans bacteria are also available from the Deutsche Sammlung von Mikroorganismen und Zellkulturen GmbH (Braunschweig, Germany) using the following accession numbers: Bacillus coagulans Hammer 1915 (DSM No. 2356); Bacillus coagulans Hammer 1915 (DSM No. 2383, corresponds to ATCC No. 11014); Bacillus coagulans Hammer (DSM No. 2384, corresponds to ATCC No. 11369); and Bacillus coagulans Hammer (DSM No. 2385, corresponds to ATCC No. 15949). Bacillus coagulans bacteria can also be obtained from commercial suppliers such as Nebraska Cultures (Walnut Creek, Calif.). Compositions include strains or variants derived from Bacillus coagulans Hammer strain ATCC No. 31284 such as ATCC PTA-6085, PTA-6086, or PTA-6087.

In some embodiments, the Bacillus coagulans is Bacillus coagulans Hammer strain Accession No. ATCC 31284, or one or more strains derived from Bacillus coagulans Hammer strain Accession No. ATCC 31284 (e.g., ATCC Numbers: GBI-20, ATCC Designation Number PTA-6085; GBI-30 (BC30), ATCC Designation Number PTA-6086; and GBI-40, ATCC Designation Number PTA-6087; see U.S. Pat. No. 6,849,256 to Farmer, the entire content of which is incorporated herein by reference).

Bacillus coagulans was previously mischaracterized as a Lactobacillus and labeled as Lactobacillus sporogenes (Nakamura et al. 1988. Int. J. Syst. Bacteria 38: 63-73). However, initial classification was incorrect because Bacillus coagulans produces spores and excretes L(+)-lactic acid through metabolism. Both of these characteristics provide key features to the utility of Bacillus coagulans. These developmental and metabolic aspects required that the bacterium be classified as a lactic acid Bacillus. In addition, it is not generally appreciated that classic Lactobacillus species are unsuitable for colonization of the gut due to their instability in the harsh (i.e., acidic) pH environment of the bile, particularly human bile. By contrast. Bacillus coagulans is able to survive and colonize the gastrointestinal tract in the bile environment and even grown in this low pH range.

### Non-Limiting Examples of Confection-Based Compositions for Stress Reduction and Prevention

Aspects of the present subject matter relate to confection compositions comprising inactivated, non-viable, or dead Bacillus coagulans bacteria or particles comprising such bacteria. The confection compositions are suitable for human or animal consumption. As used herein, a “confection” or“confection composition” includes food items that are rich in sugar or artificial sweeteners. The words “candy” or “sweets” are also used for the term “confectionery.” In various embodiments, candy may be made by dissolving sugar in water or milk to form a syrup, which is boiled until it reaches the desired concentration or starts to caramelize. In some embodiments, the type of candy depends on the ingredients and how long the mixture is boiled, while the final texture of candy depends on the sugar concentration. In certain embodiments, as the syrup is heated, it boils, water evaporates, the sugar concentration increases, and the boiling point rises. Thus, in various embodiments, boiling temperature corresponds to a particular sugar concentration. In some embodiments, higher temperatures and greater sugar concentrations result in hard, brittle candies, while lower temperatures result in softer candies. In certain embodiments, the name of a candy may come from the process used to test the syrup before thermometers became affordable; a small spoonful of syrup was dropped into cold water, and the characteristics of the resulting lump were evaluated to determine the concentration of the syrup. Long strings of hardened sugar indicate “thread” stage, while a smooth lump indicates “ball” stages, with the corresponding hardness described. The “crack” stages are indicated by a ball of candy so brittle that the rapid cooling from the water literally causes it to crack. Candy comes in an endless variety of textures from soft and chewy to hard and brittle.

There are a variety of categories and types of confections. Non-limiting examples are described herein. Hard sweets are based on sugars cooked to the hard-crack stage, including suckers, lollipops, jawbreakers (or gobstoppers), lemon drops, peppermint drops and disks, candy canes, rock candy, etc. Hard sweets also include candies often mixed with nuts, such as brittle. Others contain flavorings including coffee, such as Kopiko. Fudge is a confection of milk and sugar boiled to the soft-ball stage. Toffee (or Taffy or Tuffy) is based on sugars cooked to the soft-ball stage and then pulled to create an elastic texture. Tablet is a crumbly milk-based soft and hard candy, based on sugars cooked to the soft-ball stage, and comes in several forms, such as wafers and heart shapes. Liquorice, which contains extract of the liquorice root, is chewier and more resilient than gum/gelatin candies, but still designed for swallowing. Other types of confection include chocolates, marshmallow, marzipan, and divinity. Jelly candies include those based on sugar and starch, pectin, gum, or gelatin, e.g., jelly beans, gumdrops, jujubes, cola bottles, and gummies. In some embodiments, a jelly candy comprises a gummi candy/confection. In certain embodiments, the gummi candy may comprise a gelatin-based gummi candy. In various embodiments, the gummi candy comprises a hydrocolloid such as one or more or any combination of the following: gelatin, gellan gum, xanthan gum, pectin, carrageenan, cellulose gum, gum arabic, and modified starch. In certain embodiments, a gelatin-based gummi candy comprises at least about 5%, 10%, 15%, 20%, or 25% gelatin by weight, at least about 5%, 10%, 15%, 20%, 25%, 30%, 35%, 40%, 45%, 50%, 55%, 60%, 65%, 70%, or 75% monosaccharide or disaccharide sugar by weight, at least about 5%, 10%, 15%, 20%, 25%, 30%, 35%, 40%, 45%, or 50% starch (e.g., modified starch) or corn syrup by weight, at least about 0.1%, 0.2%, 0.3%, 0.4%, 0.5%, 0.6%, 0.7%, 0.8%, 0.9%, 1.0%, 1.1%, 1.2%, 1.3%, 1.4%, or 1.5% pectin by weight, at least about 0.1%, 0.2%, 0.3%, 0.4%, 0.5%, 0.6%, 0.7%, 0.8%, 0.9%, 1.0%, 1.1%, 1.2%, 1.3%, 1.4%, or 1.5% gellan gum by weight, and/or at least about 5%, 10%, 15%, 20%, or 25% water by weight.

Suitable gummi confections include bears, rings, worms, frogs, snakes, hamburgers, cherries, sharks, penguins, hippos, lobsters, octopuses, apples, peaches, oranges, and spiders. Suitable gummi bear sizes range from the standard candy size (or smaller), to gummi bears that weigh several kilograms. Gummi confections come in a variety of flavors, including raspberry, orange, strawberry, pineapple, and lemon.

A non-limiting example of a traditional gummi confection (e.g., gummi bears) is made from sugar, glucose syrup, starch, flavoring, food coloring, citric acid, and gelatin. Suitable gelling agents and hydrocolloids can be selected by one of ordinary skill in the art. Examples include gums, carrageenan, gelatin, pectin, high methoxy pectin, alginates, and agar. One of ordinary skill in the art can select a suitable gelling agent or hydrocolloid depending on the desired final texture of the starch molded piece. There are some gummi confections made with pectin or starch instead of gelatin, making them suitable for vegetarians. An exemplary organic gummi confection is made with most all natural ingredients, such as organic tapioca syrup, organic evaporated cane juice, gelatin, organic grape juice concentrate, citric acid, lactic acid, ascorbic acid, colors added (black, carrot juice concentrate, turmeric, annatto), natural flavors, organic sunflower oil, and carnauba wax.

In various embodiments, large sour gummi bears are larger and flatter than traditional gummi bears, have a softer texture, and include fumaric acid or other acid ingredients to produce a sour flavor. In some embodiments, sour “gummies” are produced by forming a sweet, flavored, and chewy core and subsequently dusting the exterior with a food acid, such as citric acid. In certain embodiments, the gelling ingredient in the core of these products is gelatin or pectin. In various embodiments, the acidic exterior is applied by use of a wetting agent or food adhesive. Some manufacturers produce sour bears with a different texture, based on starch instead of gelatin. Typically, starch produces a shorter (cleaner bite, less chewy) texture than gelatin.

Confection-based compositions, such as those described herein, are made from a variety of ingredients known to those skilled in the art. In some embodiments, the confection-based compositions are prepared by combining confection ingredients and a liquid, e.g., water or milk. In certain embodiments, the composition is prepared by combining confection ingredients and a liquid, and heating the resulting combination. Optionally, the combination is heated (heat-processed) using applied heat, a flame, or a microwave. In various embodiments, the confection-based composition is boiled in hot water, e.g., stovetop boiling, addition of boiling water to a container, or microwaving the confection-based composition along with water. In some embodiments, boiling water (about 100° C.) is added to a combination of confection ingredients and inactivated, non-viable, or dead Bacillus coagulans bacteria or particles comprising such bacteria.

In certain embodiments, mass production of gummi confection (e.g., gummi bears) includes mixing the gummi confection ingredients and pouring the resulting mixture into many starched-lined (e.g., corn starch-lined) trays/molds. In various embodiments, the corn starch prevents the gummy bears from sticking to the mold and lets them release easily once they are set. In some embodiments, first, the desired character molds are created and, if necessary, duplicated with a machine. Optionally, starch powder is applied to the character molds. In certain embodiments, gummi confection ingredients, such as sugar, glucose syrup, gelatin, and water are mixed together and heated. In various embodiments, the ingredients are mixed with colors and flavors that give the bears their signature look and taste. In some embodiments, the molten gelatin mixture is poured into the molds and allowed to cool and set prior to packaging or consumption. In certain embodiments, the gummi confection is subsequently heated and placed in a large drum tumbler to apply a composition of isolated inactivated, non-viable, or dead Bacillus coagulans bacteria, or particles comprising such bacteria, and a sweetener (e.g., a sugar).

In a non-limiting example, as described in WO/2009/102575, production of gummi confection includes the following: A colloid batch and a puree batch are formed and combined with corn syrup and sugar to form a base slurry. The colloid batch comprises a solution of the gelling agent in water at a level of from 5 to 15% by weight of the gelling agent, more preferably from 7 to 12% of the gelling agent based on the total weight of the colloid batch. The colloid batch is held at a temperature of 170 to 190° F. The puree batch preferably comprises water, fruit pure and/or high fructose corn syrup or other sweeteners, thin boiling starch, and sodium citrate. It is held at a temperature of from 65 to 75 F. Preferably, the fruit puree has a Brix of from 10 to 45, more preferably from 25 to 40. Optionally, the pure batch includes a plurality of fruit purees. The fruit puree comprises a typical fruit puree, a fruit juice, or a fruit powder. The puree batch comprises from 30 to 40% by weight water, from 0 to 40% by weight fruit puree, from 0 to 40% by weight high fructose corn syrup, from 25 to 35% by weight thin boiling starch, and from 0.0 to 2.0% by weight sodium citrate. In a mixing kettle from 25 to 40% by weight of additional corn syrup is combined with from 15 to 40% by weight of fine granulated sugar, from 10 to 15% by weight of the colloid batch and from 20 to 30% by weight of the puree batch to form the base slurry. Preferably, the corn syrup is approximately 42 DE corn syrup, however, as would be understood by one of ordinary skill in the art other DE corn syrups could be used. The base slurry components are completely mixed and held at 130 to 150° F. in a holding tank. The base slurry is then cooked to bring the Brix to from 70 to 85 Brix, more preferably to a Brix of from 75 to 80. In one embodiment the base slurry is passed through a coil cooker and heated to a temperature of from 250 to 325° F. to cook it. Other cooking methods will be known to those of ordinary skill in the art. In the cooked base slurry is preferably subjected to vacuum to further increase the Brix into the desired range. The cooked base slurry is held at approximately 200° F. until used. An acidulant solution is preferably added along with color and flavor to the cooked base slurry just prior to deposition in the starch molds. In one aspect, the acidulant solution comprises ascorbic acid present in an amount of from 15 to 20% by weight, citric acid present in an amount of from 10 to 20% by weight, and malic acid present in an amount of from 5 to 10% by weight with the remainder comprising water. As would be understood by one of ordinary skill in the art, other edible acids could be used in place of or in addition to those listed. In one aspect, 95 to 97% by weight of cooked base slurry is combined with from 2 to 3% by weight of the acidulant solution and the remainder comprises flavors and colors. Optionally, the acidulant solution is used to bring the pH of the base slurry to from 2.6 to 3.2. One of ordinary skill in the art would have no difficulty selecting suitable colors and flavors. The combined mixture is then deposited into starch molds, e.g., using a Mogul starch molding machine. Such starch molding machines are well known by those of ordinary skill in the art. In one aspect, from 0.3 to 3 grams of the base slurry is deposited into each mold cavity. The starch trays with deposited base slurry are transferred to a drying room where there are held for 12 to 48 hours. Optionally, the trays are first held at a temperature of from 130 to 150° F. for from 10 to 15 hours, and then cooled to 70 to 800 F and held at that temperature for from 6 to 12 hours. The gelled starch molded food pieces are then removed from the trays, the starch is recycled.

Compositions comprising chocolate and inactivated, non-viable, or dead Bacillus coagulans bacteria or particles comprising such bacteria are included herein. Chocolate has become one of the most popular food types and flavors in the world, and a vast number of foodstuffs involving chocolate have been created. Gifts of chocolate molded into different shapes have become traditional on certain holidays. Chocolate is also used in cold and hot beverages such as chocolate milk and hot chocolate.

The chocolate may be, e.g., white, plain, dark, or milk chocolate. The classification depends upon the amount of cocoa solids present in the formulation. For example, plain chocolate may have a high percentage of cocoa solids (minimum 30%, and not less than 12% dry, non-fat cocoa solids), milk chocolate may have a lower cocoa solids content (minimum 25%, and not less than 2.5% dry, non-fat cocoa solids), and white chocolate may be prepared from cocoa butter (minimum 20%) and not less than 14% milk solids. As used herein, “chocolate” includes any preparation of dry cocoa solids, non-fat cocoa solids and/or cocoa butter. Non-limiting examples of chocolate compositions include products obtained from cocoa nib, cocoa mass, cocoa, fat-reduced cocoa or any combination of two or more thereof and a sugar such as sucrose, with or without the addition of extracted cocoa butter. In some embodiments the chocolate composition contains not less than 35% total dry cocoa solids, including not less than 14% dry non-fat cocoa solids and not less than 18% permitted cocoa butter.

Non-limiting examples of chocolate compositions include coatings made from sugars, cocoa powder and/or milk solids, and/or cocoa liquor combined with vegetable fats other than cocoa butter. In various embodiments, the final chocolate formulation may be used for, e.g., coatings, molded products or panned products, and may or may not be tempered before use.

In some embodiments, a formulation may be an edible confectionery end-product in itself or may be further processed to produce such an end-product. In certain embodiments, the resulting product may be prepared for sale under ambient or low temperature conditions. Non-limiting examples of products for sale at low temperature conditions include frozen and chilled desserts, as well as confectioneries at a low temperature, such as for example of from −25° C. to +15° C., suitably from −20° C. to +5° C., to be consumed at an ambient temperature. Such low temperature products may include but are not limited to ice cream (e.g., milk- or vegetable-fat based ice cream).

In various embodiments, a chocolate formulation may also simply comprise a chocolate fat phase containing a total fat content, e.g., of at least 25% w/w prior to admixing with the concentrated sugar syrup. Suitable ranges of total fat content include. e.g., from 25% to 60% w/w, or 25% to 45% w/w, or 28% to 35% w/w. In some embodiments, such chocolate formulations are further processed into a final confectionery product. In certain embodiments, the final fat content range in the finished formulation may be at least 10% w/w or in the range of from 15% to 45% w/w or from 25% to 35% w/w. These examples should not be construed as being limiting.

Exemplary methods for formulating chocolate are provided in, e.g., European Patent No. EP 0958747B1, granted Nov. 3, 2004; U.S. Pat. No. 4,446,166, issued May 1, 1984; and U.S. Pat. No. 5,527,556, issued Jun. 18, 1996, the entire contents of each of which are incorporated herein by reference.

Confections provided herein also include “ganache” which is conventionally used as a short shelf-life filling for truffles or as a topping for confections. Ganache is the confectioner's term for a phase-inverted (i.e. oil-in-water) chocolate preparation. Ganache has a smooth, glossy texture and appearance, and a rich chocolate or milk chocolate taste. A ganache may also be produced from white chocolate in a similar way. An exemplary moisture content for ganache from 1040% w/w. In some embodiments, a ganache cannot be utilized in processing in the same way as conventional chocolate and its soft texture characteristics render it unsuitable for the majority of enrobing, molding or pan-coating operations.

In certain embodiments, a confection provided herein further comprises a sweetener (e.g., a granulated or powder sugar) coating on the exterior surface thereof, the sweeteners can comprise, e.g., one or more monosaccharides or disaccharides. Non-limiting examples include sugar, sucrose, invert sugar, dextrose, lactose, honey, malt syrup, malt syrup solids, maltose, fructose, granular fructose, maple syrup, rice syrup, rice syrup solids, sorghum syrup, refiners syrup, corn syrup, corn syrup solids, high fructose corn syrup, molasses, and any combination thereof. In some embodiments, the sugar comprises cane sugar, beet sugar, date sugar, sucanat, granulated fructose or an artificial sweetener (e.g., Sweet-n-Low®, NutraSweet®, or Equal®). Additional artificial sweeteners include acesulfame K, aspartame, sucralose, d-tagatose, and combinations thereof.

Dry Mixes and Addition of Inactivated, Non-Viable or Dead Bacillus coagulans to Food Compositions for Stress Reduction and Prevention

Compositions provided herein include a dry mix comprising inactivated, non-viable, or dead Bacillus coagulans bacteria, or particles comprising such bacteria, for inclusion within or addition to the surface of compositions. In certain embodiments, the dry mix may be between 1% and 50% inactivated, non-viable, or dead Bacillus coagulans bacteria. e.g., about 5%, about 10%, about 15%, about 20%, about 25%, about 35%, about 45%, or about 50% inactivated, non-viable, or dead Bacillus coagulans bacteria. In some embodiments, the dry mix may be about 15% inactivated, non-viable, or dead Bacillus coagulans bacteria and 85% sugar. For example, about 100 pounds of dry mix may contain about 15 pounds of inactivated, non-viable, or dead Bacillus coagulans bacteria and about 85 pounds of other edible matter such as starch or sugar.

In various embodiments, then included in a composition, the dry mix may be between about 1% and about 50% by weight of the composition, e.g., about 1% to about 20%, about 5% to about 15%; about 6%, about 7%, about 8%, about 9%, or about 10% by weight of the composition. For example, a 3 gram composition may contain about 7% dry mix by weight of the composition.

In some embodiments, inactivated, non-viable, or dead Bacillus coagulans bacteria are added directly to the composition ingredients prior to heating, molding, and subsequent cooling of the confection.

### Non-Limiting Examples of Tea Compositions for Stress Reduction and Prevention

In an aspect, a tea beverage composition comprising inactivated, non-viable, or dead Bacillus coagulans bacteria, or particles comprising such bacteria, is provided. In various embodiments, tea is a beverage made by steeping dehydrated plant matter such as leaves, buds, roots or twigs of a plant in water. In some embodiments, tea is the combination of an instant tea mix (e.g., a powder) with water. In certain embodiments, plant matter is steeped in hot water for a few (e.g. about or at least about 1, 2, 3, 4, 5, 6, 7, 8, 9, or 10 minutes, or about 1-5, 2-5, 3-5, 4-5, or 1-10 minutes). In various embodiments, a tea composition provided herein comprises dried plant matter used for making tea. In some embodiments, a tea composition provided herein comprises an instant tea mix (e.g., a dry powder). In certain embodiments, the tea is instant tea or brewable tea.

In various embodiments, the plant mater is from a Camellia sinensis plant. Non-limiting examples of tea include black tea, oolong tea, green tea, yellow tea, and white tea. In one aspect, the tea is decaffeinated tea.

In some embodiments, instant tea includes a concentrate or dehydrate of brewed tea. In certain embodiments, an instant tea formulation does not contain vegetative matter.

In various embodiments, the tea is a blend of tea. In some embodiments, a blend of tea is prepared by adding tea from different plants. e.g., a tea from a plant such as Cemellia sinensis and a plant other than Camellia sinensis. For example, the popular Earl Grey tea is black tea with bergamot, while Jasmine tea is Chinese tea with Jasmine.

In certain embodiments, a tea composition comprises herbal tea. In various embodiments, a herb is characterized as a small, seed bearing plant with fleshy, rather than woody, parts. In addition to herbaceous perennials, herbs may include trees, shrubs, annuals, vines, and more primitive plants, such as ferns, mosses, algae, lichens, and fungi. Herbs are often valued for their flavor, fragrance, medicinal and healthful qualities, economic and industrial uses, pesticidal properties, and coloring materials (e.g., as dyes). In some embodiments, a herbal tea is an infusion of vegetative matter other than from a Camellia sinensis plant. In certain embodiments, herbal tea is made with fresh or dried flowers, fruit, leaves, seeds or roots, e.g., by pouring hot (such as boiling) water over the plant parts and letting them steep for a few (e.g. about or at least about 1, 2, 3, 4, 5, 6, 7, 8, 9, or 10 minutes, or about 1-5, 2-5, 3-5, 4-5, or 1-10 minutes) minutes. In various embodiments, herbal tea is made with dried leaves, flowers, fruit, or seeds of a medicinal plant. In some embodiments, seeds and/or roots are boiled on a stove or microwaved. In certain embodiments, the herbal tea is then strained and sweetened if so desired. Non-limiting examples of herbal teas include Anise tea, roasted barley tea, Bissap tea, Cannabis tea, Catnip tea, Cerasse tea, Chamomile tea, Chrysanthemum tea (made from dried flowers), Citrus peel tea (including bergamot, lemon and orange peel), roasted corn tea. Echinacea tea, Essiac tea (a blended herbal tea), Fennel tea, Gentian tea, Ginger root tea, Ginseng tea, Greek Mountain Tea (made from a variety of the Sideritis syriaca plant), Hibiscus tea (often blended with rose hip), Honeybush tea, Horehound tea, Jiaogulan tea, Kava root tea, Labrador tea, Lapacho tea, Lemon grass tea, Licorice root tea. Lime blossom tea, Lotus flower tea, Mate tea, Mate de coca tea, Mint tea, European mistletoe tea, Neem leaf tea, Nettle leaf tea, Red raspberry leaf tea, Toasted rice tea, Rooibos (Red Bush or red) tea. Rose hip tea (often blended with hibiscus), Rosemary tea, Sage tea, Sassafras tea, Skullcap tea, Staghom Sumac tea, Stevia tea, Thyme tea, Tulsi tea, Uncaria tomentosa tea (Cats Claw), Valerian tea, Vervain tea, Vetiver tea, Roasted wheat tea. Wong Logat tea, Woodruff tea, Yarrow tea, Yuen Kut Lam Kam Wo Tea, and Tan Ngan Lo herbal tea.

In various embodiments, the tea comprises loose plant matter (e.g., the tea is not in a tea bag. In some embodiments, the tea may be placed in an infuser or strainer. In certain embodiments, the tea composition is within a tea bag. A tea bag consists of two parts, the tea and the bag. Non-limiting examples of tea bags include those of a porous silk, paper, cotton, or nylon bag with tea inside that is used for brewing tea. Inactivated, non-viable, or dead Bacillus coagulans bacteria (or particles comprising non-viable, or dead Bacillus coagulans bacteria) may be added to the tea in any way, e.g., loosely within, on or in a tea bag, and/or on plant matter (e.g., adhered to or loosely in combination with plant matter). In some embodiments, a tea bag comprises dehydrated plant matter obtained from Camellia sinensis. In some embodiments, a tea bag comprises dehydrated plant matter obtained from a plant other than Camellia sinensis. In some embodiments, the dehydrated plant matter comprises dried leaves, buds, roots, and/or twigs.

Additional non-limiting examples of non-bacterial ingredients that may be combined with inactivated, non-viable, or dead Bacillus coagulans bacteria (or particles comprising non-viable, or dead Bacillus coagulans bacteria) include coffee beans or fragments thereof, coffee powder, chocolate powder, and cocoa powder. Non-limiting examples of beverage compositions include coffee, hot chocolate, and hot cocoa. In some embodiments, the coffee is instant coffee or brewable coffee. In certain embodiments, the coffee is decaffeinated coffee. In various embodiments, a beverage composition includes a dairy product, a non-dairy creamer, a flavored creamer, a flavor extract, a natural sweetener (e.g., Stevia), or an artificial sweetener such as sucralose or granulated saccharin. In some embodiments, Inactivated, non-viable, or dead Bacillus coagulans bacteria (or particles comprising non-viable, or dead Bacillus coagulans bacteria) are in the form of spray-dried powder is added directly to the coffee (e.g. ground coffee beans or freeze-dried brewed coffee crystals or powder) itself.

### Non-Limiting Examples of Soups and Grain-Containing Compositions for Stress Reduction and Prevention

Included herein are cooked and uncooked compositions comprising a grain or a portion or processed product thereof and inactivated, non-viable, or dead Bacillus coagulans bacteria or particles comprising such bacteria. In some embodiments, the grain is an intact grain or portion thereof, e.g., a grain of a grain of wheat, a grain of rice, a grain of quinoa, a grain of fonio, a grain of barley, a grain of corn, a grain of buckwheat, a grain of rye, a grain of sorghum, a grain of millet, a grain of triticale, or a grain of teff. In certain embodiments, the grain is, e.g., husked but grain not crushed, cracked, or ground. In various embodiments, the grain is processed, e.g., altered from its naturally-occurring state. In some embodiments, the grain is husked, crushed, cracked, or ground. In certain embodiments, the grain is in the form of flour or a composition made from further manipulation of a grain-based flour. As used herein, the term “grain” includes grain-like seeds such as buckwheat. Non-limiting examples of grains include wheat, rice, quinoa, fonio, barley, corn, buckwheat, rye, sorghum, millet, triticale, and teff. Non-limiting examples of wheat include hard red winter wheat, hard red spring wheat, soft red winter wheat, soft white wheat, hart white wheat, and durum wheat. Non-limiting examples of cooked compositions include pasta, oatmeal, and grits. Non-limiting examples of pastas include egg pasta, spaghetti (solid, thin cylinders), macaroni (tubes or hollow cylinders), fusilli (spiral-shaped), lasagna (sheets), tagliatelle (flat ribbons), vermicelli (thin spaghetti), ravioli (filled pasta), spätzle, gnocchi, penne rigate (furrowed cylinder-shaped pasta), penne lisce (smooth cylinder-shaped pasta), rotini (corkscrew-shaped pasta), and rigatoni (tube-shaped pasta).

In some embodiments, the composition comprises a dry mix grain-based composition comprising a grain and inactivated, non-viable, or dead Bacillus coagulans bacteria or particles comprising such bacteria. Also provided are compositions comprising a dry mix for soup comprising a dehydrated matter and inactivated, non-viable, or dead Bacillus coagulans bacteria or particles comprising such bacteria.

Also provided are methods of making a grain-based composition comprising providing a grain-containing base mix and a liquid portion; mixing the grain-containing base mix and the liquid portion to form a batter or dough; combining viable Bacillus coagulans with the batter or dough; and heat processing the batter or dough at a temperature that kills all or substantially all of the viable Bacillus coagulans to cook the grain-based composition. In various embodiments, the liquid portion include water or milk. In some embodiments, the viable Bacillus coagulans is in the form of a spore. In some embodiments, the viable Bacillus coagulans is in the form of a vegetative cell.

Non-limiting examples of grain-based compositions include pasta, oatmeal, grits, and cereal. Common (non-limiting) varieties of pasta include tubular pasta, straight round rod pasta, ribbon pasta, micro pasta, stuffed pasta, irregular-shaped pasta, spaghetti (solid, thin cylinders), macaroni (tubes or hollow cylinders), fusilli (spiral-shaped), lasagna (sheets), tagliatelle (flat ribbons), vermicelli (thin spaghetti), and ravioli (filled pasta), penne (cylinder-shaped pasta), rotini (corkscrew-shaped pasta), rigatoni (tube-shaped pasta), noodles, and spätzle. In some embodiments, the pasta is dried. In certain embodiments, the pasta is fresh. In various embodiments, the pasta includes egg (egg pasta). In some embodiments, the pasta does not include egg.

Many ingredients may be used to make pasta dough, ranging from a simple flour and water mixture, to those that call for the addition of eggs, spices and cheeses, or even squid ink to the dough. In certain embodiments, the pasta contains a filling, e.g., cheese, vegetables, fruit, and/or meat. In various embodiments, dry pasta is made from durum wheat flour, farina flour, or semolina flour. Some pasta varieties, such as pizzoccheri, are made from buckwheat flour.

In some embodiments, a composition provided herein comprises gnocchi [often considered to be pasta, although it can have quite different ingredients (such as milled potatoes)].

Also provided are grain-based compositions in the form of oatmeal with inactivated, non-viable, or dead Bacillus coagulans bacteria or particles comprising such bacteria. Oatmeal is a product of ground oat groats (e.g., oatmeal, cornmeal, peasemeal, etc.) or a porridge made from this product (also called oatmeal cereal). In some embodiments, oatmeal includes other products made from oat groats, such as cut oats, crushed oats, and rolled oats. In certain embodiments, the groats are coarsely ground to make oatmeal, or cut into small pieces to make steel-cut oats, or steamed and rolled to make rolled oats. In various embodiments relating to rolled oats, oat groats are steamed, pressed with a roller, and dried. In some embodiments, the oatmeal is instant oatmeal. In certain embodiments, instant oatmeal is pre-cooked and dried. In various embodiments, the oatmeal includes a sweetener and/or a another ingredient (such as an ingredient that adds flavor). Non-limiting examples of sweeteners and flavor additives include salt, white sugar, brown sugar, stevia, cinnamon, honey, jam, molasses, maple syrup, butter, chocolate, soy sauce, soy milk, milk, vinegar, condensed or evaporated milk, and cream. In some embodiments, one or more fruits and/or nuts are added, such as strawberries, blueberries, apples, peaches, mangos, bananas, raisins, dried cherries, dried cranberries, pecans, walnuts, and peanut butter. In certain embodiments, oatmeal is used to make porridge, as an ingredient (as in oatmeal cookies and oat cakes), or as an accent as in the topping on an oat bran bread or as the coating on caboe cheese. In various embodiments, oatmeal is used as a thickener in a food such as canned chili con came. In some embodiments, oatmeal is used in an animal feed product.

In certain embodiments, the composition comprises grits and inactivated, non-viable, or dead Bacillus coagulans bacteria, or particles comprising such bacteria, are also provided.

Also provided are soups, such as those that are cold and those that are hot. In various embodiments, soup is a food that is made by combining ingredients such as meat and vegetables in stock or hot/boiling water, until the flavor is extracted, forming a broth. Optionally, soups may be classified into two broad groups: clear soups and thick soups. Thick soups are classified depending upon the type of thickening agent used: purees are vegetable soups thickened with starch; bisques are made from pureed shellfish thickened with cream; cream soups are thickened with bechamel sauce; and veloutes are thickened with eggs, butter and cream. Other ingredients commonly used to thicken soups and broths include rice, flour, and grain. In some embodiments, mixes containing ramen noodles are marketed as an inexpensive instant lunch, requiring only hot water for preparation. Non-limiting types of soups include tomato soup, cream of mushroom soup, chicken noodle soup, vegetable beef soup, minestrone soup, leek and potato soup, lentil soup, fish soup, miso soup, pea soup, fruit soup, clam chowder, gumbo, and bisque. In certain embodiments, a soup, such as vegetable, chicken base, potato, pasta and cheese soups, are available in dry mix form, ready to be served by adding hot water. In various embodiments, a dry mix soup includes dehydrated matter, e.g., dehydrated meat, such as poultry and beef, dehydrated vegetables, dehydrated herbs, dehydrated spices, and/or dehydrated noodles. In some embodiments, a packet of dry soup stock (e.g., ramen) does not contain water. In certain embodiments, an instant soup is preserved into a dry powder which can be stored in, e.g., a packet or a cup. In some embodiments, the inactivated, non-viable, or dead Bacillus coagulans bacteria, or particles comprising such bacteria, are in the form of a powder that is added prior to or subsequent to addition of the dry soup mix to hot water. In certain embodiments, inactivated, non-viable, or dead Bacillus coagulans bacteria, or particles comprising such bacteria, are within the dry soup mix.

In various embodiments, a composition is a baked composition that comprises inactivated, non-viable, or dead Bacillus coagulans bacteria or particles comprising such bacteria. Non-limiting examples of baked compositions include a bread, a cake, a muffin, a pie, a tart, a pastry, a food bar, a granola bar, a quiche, a cookie, a pizza, a baked corn chip, a baked tortilla chip, a baked potato chip, a baked cracker, and baked treats for companion animals. In some embodiments, a baked composition comprises flour. In certain embodiments, a baked composition is a good that is heated. e.g., baked (exposure of dry heat).

In certain embodiments, a baked composition includes a fat. Non-limiting examples of fats include oils, butters, shortenings, artificial lipids, and synthetic fats. Alternatively or in addition a baked composition comprises a fat substitute. In certain embodiments, a baked composition also includes a sugar, or a sugar substitute. In various embodiments, a baked composition comprises an artificial sweetener.

In some embodiments, the composition is a dry mix for a baked composition including a flour and inactivated, non-viable, or dead Bacillus coagulans bacteria or particles comprising such bacteria.

In certain embodiments, the composition is bread that contains inactivated, non-viable, or dead Bacillus coagulans bacteria or particles comprising such bacteria. Any method of making bread may be used. In various embodiments, bread includes flour and water. In some embodiments, salt is also present. In certain embodiments, a leavening agent tis a biological leavening agent, such as yeast, or a chemical leavening agent, such as, baking powder, or baking soda is used. Non-limiting examples of flour include wheat flour, rice flour, corn flour, rye flour, potato flour, millet flour, baking flour, graham flour, and quinoa flour. In various embodiments, the flour is self-rising or self-rising flour. In some embodiments, bread also contains an amount of sugar, spices, fruit (such as raisins, pumpkins, bananas, strawberries, blueberries, and the like), vegetables (such as onion or zucchini, and the like), nuts, or seeds (such as caraway, sesame or poppy seeds). In some embodiments, an oil (vegetable oil, corn oil, olive oil, grape seed oil, nut oil or fruit oil), butter, shortening, artificial lipid, synthetic fat, or a fat substitute such as olestra is also present. In certain embodiments, a sugar, sugar substitute, or artificial sweetener such as saccharin, sucralose or aspartame is present. Non-limiting examples of baked compositions include, but are not limited to, buns, rolls, bagels, cookies, and pastries.

In various embodiments, the inactivated, non-viable, or dead Bacillus coagulans bacteria, or particles comprising such bacteria, are impregnated into the baked composition during the manufacturing process of the baked composition (e.g., added to the batter or dough mix). In some embodiments, the inactivated, non-viable, or dead Bacillus coagulans bacteria, or particles comprising such bacteria, are added to i.e., present on the exterior, of the baked composition (e.g., as a coating on at least a portion of the exterior surface of the baked composition).

### Non-Limiting Examples of Non-Dairy Milk-Like Compositions for Stress Reduction and Prevention

In certain embodiments, the composition is a non-dairy milk-like composition containing inactivated, non-viable, or dead Bacillus coagulans bacteria or particles comprising such bacteria. Such compositions may provide probiotic benefit to subjects who are vegans, desire a decreased milk cholesterol content, are lactose intolerant, exhibit allergies towards milk proteins, or cannot tolerate or do not wish to consume animal products or by-products.

In various embodiments, a dairy product is a food or drink product made from or containing the milk of a mammal such as a cow, sheep, or goat. A “milk-like composition” does not contain the milk of a mammal. In some embodiments, a milk-like composition has an appearance and/or texture of cow's milk. In certain embodiments, a milk-like composition comprises a liquid from a pressed or pulverized flower, seed, grain, nut, or legume. In various embodiments, a milk-like composition is produced from peas, peanuts, lentils, beans (e.g., soy beans), almonds, cashews, pecans, macadamias, hazelnuts, walnuts, barley, oats, rice, spelt, hemp seeds, pumpkin seeds, quinoa, lupines, sesame seeds, sunflower seeds, and/or coconuts.

In some embodiments, the composition comprises a non-dairy milk-like composition such as milk, cheese, yoghurt, ice cream, pudding, cream cheese, sour cream, coffee creamer, kefir, cottage cheese or mayonnaise. In certain embodiments, a non-dairy milk-like composition includes inactivated, non-viable, or dead Bacillus coagulans bacteria, or particles comprising such bacteria, combined with a non-dairy milk-like composition, such as those made from plantmilk, which can be derived from grains (barley, oat, rice, spelt), legumes (peas, peanuts, lentils, beans, soy), nuts (almonds, cashews, pecans, macadamias, hazelnuts, walnuts), and seeds (hemp, pumpkin, quinoa, lupines, sesame, pumpkin, sunflower, coconut).

Non-Limiting Examples of Compositions and Uses for Reducing Stress Associated with Exercise

Included herein are sports nutrition compositions comprising inactivated, non-viable, or dead Bacillus coagulans bacteria or particles comprising such bacteria. In various embodiments, the sports nutrition compositions help reduce stress associated with physical exhertion such as exercise. In some embodiments, sports nutrition compositions comprise a large amount of calories per unit dose to assist a subject in gaining weight, e.g., muscle weight. A unit dose of the compositions described herein is the amount of composition administered to a consumer in a single dose, i.e., one serving. Unit-dose packaging is the packaging of a single dose, e.g., in a non-reusable container. For example, a unit dose refers to a physically discrete unit suitable as unitary doses for an individual, each unit containing a predetermined quantity of active material calculated to produce the desired effect, in association with a suitable carrier, diluent, or excipient. In certain embodiments, packaging may include, e.g., single or multiple unit dosages.

Compositions that provide a large amount of calories to assist a subject in gaining weight comprise compositions comprising between about 100 and about 10,000 food Calories (kcal) per unit dose (i.e., serving). e.g., between 250 and 5,000 kcal, between 500 and 3,000 kcal, between 750 and 2,500 kcal, or between 1,000 and 2,000 kcal, e.g., about 1,000 kcal, about 1,100 kcal, about 1,200 kcal, about 1,300 kcal, about 1,400 kcal, about 1,500 kcal, about 1,600 kcal, about 1,700 kcal, about 1,800 kcal, about 1,900 kcal, or about 2,000 kcal.

In some embodiments, a composition does not comprise a large amount of calories. In certain embodiments, a composition comprises between about 10 and 500 kcal, e.g., between about 20 and 250 kcal, between about 50 and 200 kcal, or between about 100 and 150 kcal, e.g., about 100 kcal, about 110 kcal, about 120 kcal, about 130 kcal, about 140 kcal, or about 150 kcal.

In certain embodiments, a composition comprises protein. For example, the protein comprises about 1% to about 99% by weight of the composition, e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% by weight of the composition. For example, the composition comprises between 1 gram and 500 grams of protein, e.g., about 10 grams, about 15 grams, about 20 grams, about 25 grams, about 30 grams, about 35 grams, about 40 grams, about 45 grams, about 50 grams, about 55 grams, about 60 grams, about 65 grams, about 70 grams, about 75 grams, about 80 grams, about 85 grams, about 90 grams, about 95 grams, about 100 grams, about 150 grams, about 200 grams, about 250 grams, about 300 grams, about 350 grams, about 400 grams, about 450 grams, or about 500 grams of protein. In various embodiments, a sports nutrition composition comprises purified or processed protein, such as soy protein, whey protein, rice protein, hemp seed protein, casein protein, or milk protein. In some embodiments, the composition comprises an amino acid selected from the group consisting of isoleucine, alanine, leucine, arginine, lysine, aspartate, aspartic acid, methionine, cysteine, phenylalanine, threonine, tryptophan, glycine, valine, proline, histidine, serine, tyrosine, asparagine, selenocysteine, pyrrolysine, glutamate, glutamic acid, and glutamine.

In certain embodiments, a sports nutrition composition comprises creatine, calcium, sodium caseinate, a whey peptide, or lactoferrin.

In various embodiments, a composition comprises an ingredients such as sodium, potassium, sugar, carbohydrates, dietary fiber, vitamin A, vitamin C, calcium, iron, vitamin D, vitamin E, thiamin, riboflavin, niacin, vitamin B6, folate, vitamin B12, biotin, pantothenic acid, phosphorus, iodine, magnesium, zinc, selenium, copper, manganese, chromium, or molybdenum. In some embodiments, a composition comprises a glucose polymer, a protein blend (e.g., whey protein concentrate, whey protein isolate, egg albumin, milk protein isolate, and partially hydrolyzed whey protein), rice protein concentrate, brown rice concentrate, taurine, L-glutamine, non-dairy creamer (e.g. sunflower oil, a corn syrup solid, sodium caseinate, a monoglyceride, a diglyceride, dipotassium phosphate, tricalcium phosphate, soy lecithin, and/or a tocopherol), a natural and artificial flavor, xantham gum, calcium citrate, potassium citrate, dipotassium phosphate, cellulose gum, tricalcium phosphate, magnesium aspartate, rice starch, carrageenan, a vitamin or mineral (e.g., ascorbic acid, niacinamide, d-alpha tocopheryl succinate, d-calcium pantothonate, zinc citrate, pyridoxine hydrochloride, ferrous fumarate, thiamine mononitrate, riboflavin, manganese amino acid chelate, beta-carotene, copper gluconate, folic acid, biotin, potassium iodide, chromium polynicotinate, molybdenum amino acid chelate, selenomethionine, cyanocobalamin, and/or cholecalciferol), sucralose, acesulfame potassium, and/or lactase.

In certain embodiments, a sports nutrition composition comprises an inactive ingredient such as an excipient, binder, or filler. Fillers fill out the size of the compositions, making it practical to produce and convenient for a subject to use. In various embodiments, by increasing the bulk volume, the fillers make it possible for the final product to have the proper volume for handling by an individual. Non-limiting examples of fillers include xantham gum, cellulose gum, lecithin, lactose, sucrose, glucose, mannitol, sorbitol, calcium carbonate, and magnesium stearate.

In some embodiments, a composition does not comprise certain ingredients. In certain embodiments, the composition does not include a sugar (e.g., glucose, fructose, galactose, maltose or lactose), gluten, aspartame, and/or artificial coloring.

In certain embodiments, a composition comprises protein powder, a ready to drink protein shake, a protein bar, a protein bite, or a protein gel.

In various embodiments, a composition is a dry mix sports nutrition composition comprising inactivated, non-viable, or dead Bacillus coagulans bacteria or particles comprising such bacteria. In some embodiments, the dry mix includes soy protein, whey protein, rice protein, hemp seed protein, and/or casein protein. In certain embodiments, a sports nutrition composition includes creatine, calcium, sodium caseinate, whey peptides, and/or lactoferrin.

In some embodiments, a sports nutrition composition includes a protein, amino acid such as branched-chain amino acid (BCAA), glutamine, essential fatty acid, meal replacement product, prohormone, creatine, thermogenic product, and/or testosterone booster. BCAAs include leucine, isoleucine, and valine.

Protein products may come in various forms, including protein powder, and ready to drink protein shakes, bars, bites, and gels. In certain embodiments, a protein product may have a flavor such as pineapple, orange, fruit punch, mixed berry, mango, cookies and cream, strawberry, strawberry banana, French vanilla, vanilla, vanilla ice cream, vanilla milkshake, banana, banana cream. Dutch chocolate, mocha cappuccino, double rich chocolate, chocolate caramel, chocolate milkshake, extreme milk chocolate, chocolate mint, chocolate chip, and chocolate. In various embodiments, protein powder is mixed with water, milk or juice (e.g., grapefruit juice, grape juice, and orange juice), resulting in a form known as a “protein shake” (as in milkshake) or “pudding.”

In some embodiments, the composition is a meal replacement product (MRP) comprising inactivated, non-viable, or dead Bacillus coagulans bacteria or particles comprising such bacteria. In certain embodiments, MPRs are either pre-packaged powdered drink mixes or edible bars designed to replace prepared meals. In various embodiments, a MRP is high in protein, low in fat, has a low to moderate amount of carbohydrates, and contains a wide array of vitamins and minerals. In some embodiments, a MRP uses whey protein, casein (e.g., calcium caseinate or micellar casein), soy protein, and/or egg albumin as a protein source. In certain embodiments, a carbohydrate is derived from maltodextrin, oat fiber, brown rice, and/or wheat flour. In various embodiments, a compositions such as MRPs comprise flax seed oil.

In various embodiments, a sports nutrition composition provided herein comprises a bodybuilding ingredient such as calcium, sodium caseinate, whey peptide, a glutamine peptide, L-glutamine, calcium alpha-ketoglutarate, isolated/free amino acids, lactoferrin, conjugated linoleic acid, medium chain triglycerides, or creatine (e.g., creatine monohydrate).

In some embodiments, sports nutrition composition ingredients are blended together as dry ingredients.

In certain embodiments, a sports nutrition composition is ready for immediate use or for storage in a sterile package. e.g., a 3-ounce package (e.g., a bag or a bottle), a 6-ounce package, a 9-ounce package, a 12-ounce package, a 15-ounce package, an 18-ounce package, a 24-ounce package, a 48-ounce package, 80-ounce package, or 100-ounce package. In various embodiments, a dried powder is packaged in unit dose quantities, e.g., 5 grams, 10 grams, 20 grams, 30 grams, 40 grams, 50 grams, 60 grams, 70 grams, 80 grams, 90 grams, or 100 gram packets. In some embodiments, a dried powder is packaged in bulk, e.g., about 500 grams, about 600 grams, about 700 grams, about 800 grams, about 900 grams, about 1,000 grams, about 1,250 grams, about 1,500 grams, about 1,750 grams, about 2,000 grams, about 2,250 grams, about 2,500 gram, or about 3,000 gram containers. In certain embodiments, the sports nutrition composition is stored in a sterile package at room temperature prior to consumption.

### Non-Limiting Examples of Oil and Fatty Acid Compositions for Stress Reduction and Prevention

Also included herein are compositions comprising an omega-3 fatty acid and inactivated, non-viable, or dead Bacillus coagulans bacteria, or particles comprising such bacteria. In various embodiments, the omega-3 fatty acid comprises eicosapentaenoic acid or docosahexaenoic acid. In some embodiments, the omega-3 fatty acid has been produced by microalgae. In certain embodiments, the omega-3 fatty acid is in oil. In various embodiments, the oil comprises seafood oil. In some embodiments, the oil comprises shellfish oil or fish oil. In certain embodiments, the oil comprises krill oil. In various embodiments, the oil comprises salmon oil, cod oil, herring oil, anchovy oil, sardine oil, or pollock oil, tuna oil, catfish oil, flounder oil, lake trout oil, grouper oil, halibut oil, mahi mahi oil, orange roughy oil, red snapper oil, shark oil, swordfish oil, tilefish oil, or mackerel oil. In some embodiments, the oil comprises cod oil, such as cod liver oil.

In certain embodiments, the composition is encapsulated in a soft-shelled capsule or a soft gelatin capsule. In various embodiments, the oil has been processed to remove an impurity (such as a toxin, polychlorinated biphenyl, or mercury). In some embodiments, the inactivated, non-viable, or dead Bacillus coagulans bacteria, or particles comprising such bacteria, and an oil are encapsulated together.

In certain embodiments, the composition comprises a preservative.

In various embodiments, the omega-3 fatty acid comprises eicosapentaenoic acid and/or docosahexaenoic acid.

In some embodiments, the oil is converted to ethyl esters. In certain embodiments, the oil is subjected to trans-esterification. In various embodiments, the oil is subjected to molecular or vacuum distillation to remove other fats and undesirable elements and to concentrate the oil. In some embodiments, an ingredient such as acid clay is added to remove a pungent smell from fish oil.

### Stress

Physiological stress exists in two varieties: 1) psychological or mental, e.g., caused by worry, fear, and/or anxiety, and 2) physical, e.g., in response to exertion, confinement, and/or general body discomfort. Regardless of the source of stress, it affects the body through the same mechanism, increasing heart rate, cortisol, blood pressure and/or epinephrine production. The stress response sereves or involuntary serves the purpose of allowing escape from danger or for producing the necessary energy to complete a hard task. Prolonged stress may have negative effects. It has been linked to mental illness, heart problems, immune system deficiencies, accelerated cell aging, oral health problems, anxiety, depression, and other serious diseases.

Stress can be defined as the dominance of the sympathetic branch of the autonomic nervous system (ANS) over the parasympathetic branch. Sympathetic activity results in physiological changes that are associated with stressful situations: increased heart rate, vasoconstriction, increased blood pressure. Parasympathetic activity has the opposite effect and is generally highest during rest.

Autonomic nervous system function can be measured in a variety of ways. A direct method would involve placing microelectrodes in the vicinity of the vagus and the sympathetic nerves in order to record the electrical activity of both of these autonomic nervous system branches. This method suffers from the highly invasive procedure and difficult implementation, both of which make it impractical for use on human subjects. Sarbach et al. (U.S. Pat. No. 7,049,149) describe an alternative method wherein the subject's stress state is estimated via chemical analysis of his or her exhalation. A similar analysis could be done on the subject's blood, looking for certain hormones that are released by sympathetic activity (cortisol, adrenaline, etc.). Sympathetic and parasympathetic activity can also be estimated by looking at the frequency spectrum of a long sequence of a hearts inter-beat intervals (Akselrod, S., et al., Power spectrum analysis of heart rate fluctuation: a quantitative probe of beat-to-beat cardiovascular control. Science, 1981 213(4504): p. 220-2).

Physiologically, the interpretation of a situation as being stressful leads to the activation of the hypothalamic-pituitary-adrenal (HPA) axis, and to the ultimate secretion of cortisol and catecholamines in humans. The end products of HPA activation (cortisol and catecholamines) are measurable in blood, urine and saliva.

Stressful situations trigger the activation of the hypothalamic-pituitary-adrenal (HPA) axis whereby neurons in the hypothalamus, a brain structure often termed the “master gland”, releases a hormone called corticotropin-releasing hormone (CRH). The release of CRH triggers the subsequent secretion and release of another hormone called adrenocorticotropin (ACTH) from the pituitary gland, also located in the brain. When ACTH is secreted by the pituitary gland, it travels in the blood and reaches the adrenal glands, which are located above the kidneys, and triggers secretion of the so-called stress hormones. As discussed above, there are two main stress hormones, the glucocorticoids (called corticosterone in animals, and cortisol in humans), and the catecholamines (epinephrine and norepinephrine). Under normal (non-stressed) conditions, cortisol secretion shows pronounced circadian rhythmicity, where concentrations are at their highest in the morning (the circadian peak), progressively decline from late afternoon to early nocturnal periods (the circadian trough), and show abrupt elevations after the first few hours of sleep. The acute secretion of glucocorticoids and catecholamines constitutes the primary mediators in the chain of hormonal events triggered in response to stress. When these two hormones are secreted in response to stress, they act on the body to give rise to the fight-or-flight response whereby one would, for instance, experience an increase in heart rate and blood pressure.

Cortisol measured in saliva reflects the fraction of cortisol that is “free” or “unbound” (to carrier proteins), the portion that crosses the blood-brain-barrier to affect different brain structures. This mechanism is believed to be at the basis for alterations in higher-order cognitive functions and behavior. This free fraction of cortisol, after crossing the blood-brain-barrier, binds to receptors in brain structures that are known to be involved in learning, memory, and emotional processing (for a review, see Lupien et al., (2005). Stress hormones and human memory function across the lifespan. Psychoneuroendocrinology, 30(3), 225-242).

Many assay techniques are available to quantify free cortisol from saliva samples. The most common assays are radioimmunoassay (RIA), time-resolved immunoassay with fluorometric detection (DELFIA) and enzyme immunoassay (EIA). These techniques rely on the principle of competitive binding between free cortisol and reagents. Correlations between concentrations yielded from these techniques depend on the type of population tested (clinical vs. healthy) and on the range in concentrations assayed (Raff. H., Homar. P. J., & Burns, E. A. (2002). Comparison of two methods for measuring salivary cortisol. Clin Chem. 48(1), 207-208; Raff, H., Homar, P. J., & Skoner, D. P. (2003). New enzyme immunoassay for salivary cortisol. Clin Chem, 49(1), 203-204).

In certain embodiments, a subject's stress levels are measured by taking samples over periods of time and measuring the cortisol levels. Compositions embodied herein are administered to the subjects, before, during and/or after an event which induces stress, for example, as described in the examples section, wherein the cortisol levels measured at varying times.

### Kits, Packaging

In certain embodiments, the compositions embodied herein are packaged in containers, e.g. bottles, boxes, foil wrapped etc., with inscriptions or inserts providing instructions to the consumer regarding the unit amounts per serving and the ingredients of the compositions. Accordingly, packaged products (e.g., sterile containers containing one or more of the compositions described herein and packaged for storage, shipment, or sale at concentrated or ready-to-use concentrations) and kits, including at least one composition of the invention and instructions for use, are also within the scope of the invention. A product can include a container (e.g., a vial, jar, bottle, bag, or the like) containing one or more compositions of the invention. In addition, an article of manufacture further may include, for example, packaging materials, instructions for use, delivery devices, buffers or other control reagents for treating or preventing the condition for which prophylaxis or treatment is required. The product may also include a legend (e.g., a printed label or insert or other medium describing the product's use (e.g., an audio- or videotape)). The legend can be associated with the container (e.g., affixed to the container) and can describe the manner in which the compositions therein should be administered (e.g., the frequency and route of administration), indications therefor, and other uses. The compositions can be ready for administration (e.g., present in dose-appropriate units), and may include one or more additional carriers or other diluents and/or an additional ingredients. Alternatively, the compositions can be provided in a concentrated form with a diluent and instructions for dilution.

In certain embodiments, the unit dose or amount comprises inactivated, non-viable, or dead Bacillus coagulans bacteria to treat or prevent stress or anxiety in a subject.

The form of administration of the inactivated probiotic in the method of the invention is not critical. In certain embodiments, the composition is in the form of a tablet, a capsule, a powder, a suspension, an aqueous solution, a food, or a beverage. In some embodiments, the inactivated, non-viable, or dead Bacillus coagulans bacteria (or particles comprising inactivated, non-viable, or dead Bacillus coagulans bacteria) are administered to a subject via tablets, pills, encapsulations, caplets, gel caps, capsules, oil drops, or sachets. In certain embodiments, the inactivated, non-viable, or dead Bacillus coagulans bacteria or particles, are encapsulated in a sugar, fat, or polysaccharide. In various embodiments, inactivated, non-viable, or dead Bacillus coagulans bacteria or particles, are added to a food or drink product and consumed. In some embodiments, the food or drink product is a nutritional product for children such as a follow-on formula, growing up milk, beverage, milk, yogurt, fruit juice, fruit-based drink, chewable tablet, cookie, cracker, or a milk powder. In certain embodiments, the product is an infant nutritional product, such as an infant formula or a human milk fortifier. In some embodiments, the edible composition comprises a hard sweet, fudge, toffee, liquorice, chocolate, jelly candy, marshmallow, and marzipan. In various embodiments, the edible composition comprises chocolate. For example, the edible composition may include a candy bar comprising chocolate and at least one other ingredient. In certain embodiments, the product is a sports nutrition composition. In some embodiments, the edible composition is a beverage. In certain embodiments, the edible composition is an alcoholic beverage. In various embodiments, the edible composition is a fermented food or beverage. In some embodiments, the edible composition comprises a soup. In certain embodiments, the edible composition comprises a grain. In various embodiments, the edible composition comprises a grain-based composition. In some embodiments, the grain-based composition comprises pasta, oatmeal, grits, or cereal. In certain embodiments, the edible composition comprises a baked composition. In some embodiments, the composition comprises a dairy composition. In certain embodiments, the composition comprises a milk-like composition.

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1: Preparation of Bacillus coagulans Cultures

Bacillus coagulans Hammer bacteria (ATCC Accession No. 31284) was inoculated and grown to a cell density of about 10⁸ to 10⁹ cells/ml in nutrient broth containing 5 g Peptone, 3 g Meat extract, 10-30 mg MnSO₄, and 1.000 ml distilled water, adjusted to pH 7.0, using a standard airlift fermentation vessel at 30° C. The range of MnSO₄ acceptable for sporulation is 1 mg/l to 1 g/l. The vegetative cells can actively reproduce up to 45° C. After fermentation, the B. coagulans bacterial cells or spores are collected using standard methods (e.g., filtration, centrifugation) and the collected cells and spores can be lyophilized, spray-dried, air-dried, or frozen. The supernatant from the cell culture is collected and used as an extracellular agent secreted by B. coagulans.

A typical yield from the above culture is in the range of about 109 to 10¹⁰ viable spores and more typically about 100 to 150 billion cells/spores per gram before drying. Spores maintain at least 90% viability after drying when stored at room temperature for up to ten years, and thus the effective shelf life of a composition containing B. coagulans Hammer spores at room temperature is about 10 years.

### Example 2: Preparation of Bacillus coagulans Spores

A culture of dried B. coagulans spores was prepared as follows. Ten million spores were inoculated into a one liter culture containing 24 g potato dextrose broth, 10 g of enzymic-digest of poultry and fish tissue, 5 g of FOS and 10 g MnSO₄. The culture was maintained for 72 hours under a high oxygen environment at 37° C. to produce culture having about 150 billion cells per gram of culture. Thereafter, the culture was filtered to remove culture medium liquid, and the bacterial pellet was resuspended in water and freeze-dried. The freeze-dried powder is then ground to a fine powder using standard good manufacturing practice (GMP).

### Example 3: Safety and Efficacy of a Dietary Ingredient in Supporting the Immune System at Rest and in Response to Both a Bacterial (LPS) Challenge and Stressful Exercise

In this 16-subject study, STAIMUNE™ was dosed (50 mg per day) and taken daily for one month (28 days). STAIMUNE™ comprises dead, non-proliferative, or inactivated Bacillus coagulans, e.g., heat-killed Bacillus coagulans. The goal of the study was to learn how STAIMUNE™ affects resting immunity (without a challenge) as well as after induced stress and to see how STAIMUNE™ may impact overall stress response. Stress in this study included directed supervised hard exercise plus being exposed to a bacterial challenge (two stressors). STAIMUNE™ was found to support mucosal immunity (immune system response of the mucous membranes ie oral, gut, and lung surfaces). STAIMUNE™ was found to support a healthy stress response. STAIMUNE™ was found to support normal inflammatory response to stress. Most importantly. STAIMUNE™ was found to reduce the body's overall stress response, while maintaining immunity. STAIMUNE™ helped significantly reduce Cortisol (hormone released during times of stress) by ˜30% in response to stress. This helps avoid negative effects of stress. Overall, it appears that STAIMUNE™ may have “adaptogenic” effects (improves body's ability to adapt to challenges including stress).

In the tables below, any underlined and italicized numbers indicate Statistical Significance (p<0.05), whereas any underlined but not italicized numbers equal Statistical Trend for difference (p>0.50 through 0.10).

TABLE 1
Subject Disposition

| Population | Overall Study | |
|---|---|---|
| Total Number of Subjects Screened | 39 | |
| Total Number of Subjects Randomized | 16 | |
| Total Number of Subjects Enrolled | 16 | |
| Safety Population | 16 | (100%) |
| Intent-to-Treat Population | 16 | (100%) |
| Per-Protocol (PP) Population | 14 | (87.5%) |

### Interpretation:

A total of 39 subjects were screened; 16 subjects entered the study and were randomized 3:1 to have 12 in the STAIMUNE™ Product Group and 4 in the Placebo group. Of the 16 subjects, 14 subjects completed all of the study visits, tests, and procedures.

TABLE 2
Protocol Deviations

| | | | | | | SOP |
|---|---|---|---|---|---|---|
| | | Time | | | Major/Minor | Deviation/Protocol |
| Subject | Visit | Point | Product | Deviation | Violation | Deviation |
| 14 | 2 | 647.5 hr | A | Blood draw was plus | Minor | SOP |
| | | | (STAIMUNE ™) | 3 minutes late due | | |
| | | | | to difficult draw | | |

### Interpretation:

The above stated deviation was minor and there will be no impact on the study outcome.

TABLE 3
Baseline Characteristics of Subjects in the Safety Population

| | | Active Product | Placebo | | Total Subjects | Reference |
|---|---|---|---|---|---|---|
| Variable | Statistic | (N = 12) | (N = 4) | P-Value | (N = 16) | Range |
| Age years | Mean ± SD | 22.00 ± 2.98 | 24.50 ± 4.20 | 0.3320 | 22.63 ± 3.36 | 18-30 |
| | Median (Min-Max) | 21.00 | 24.50 | | 21.50 | |
| | C.I | (19.00-29.00) | (20.00-29.00) | | (19.00-29.00) | |
| | | 20.10-23.90 | 17.81-31.19 | | 20.83-24.42 | |
| Weight kg | Mean ± SD | 83.57 ± 12.55 | 87.70 ± 11.66 | 0.5709 | 84.60 ± 12.09 | |
| | Median (Min-Max) | 81.95 | 88.25 | | 81.95 | |
| | C.I | (64.00-104.90) | (74.70-99.60) | | (64.00-104.90) | |
| | | 75.59-91.54 | 69.15-106.25 | | 78.16-91.04 | |
| Height cm | Mean ± SD | 181.75 ± 7.85 | 179.50 ± 4.80 | 0.5128 | 181.19 ± 7.13 | |
| | Median (Min-Max) | 178.00 | 178.50 | | 178.00 | |
| | C.I | (172.00-193.00) | (175.00-186.00) | | (172.00-193.00) | |
| | | 176.76-186.74 | 171.87-187.13 | | 177.39-184.99 | |
| Body Mass Index | Mean ± SD | 25.24 ± 2.78 | 27.13 ± 2.54 | 0.2600 | 25.71 ± 2.77 | 18.0 to 34.9 |
| (BMI) kg/m² | Median (Min-Max) | 25.60 | 27.65 | | 26.10 | |
| | C.I | (20.70-28.20) | (23.80-29.40) | | (20.70-29.40) | |
| | | 23.48-27.00 | 23.08-31.17 | | 24.24-27.19 | |
| Systolic BP | Mean ± SD | 119.50 ± 11.49 | 116.25 ± 8.26 | 0.5582 | 118.69 ± 10.61 | |
| mm Hg | Median (Min-Max) | 121.50 | 116.50 | | 119.00 | |
| | C.I | (103.00-134.00) | (106.00-126.00) | | (103.00-134.00) | |
| | | 112.20-126.80 | 103.10-129.40 | | 113.03-124.34 | |
| Diastolic BP | Mean ± SD | 75.33 ± 10.16 | 75.75 ± 6.02 | 0.9232 | 75.44 ± 9.11 | |
| mm Hg | Median (Min-Max) | 77.00 | 77.00 | | 77.00 | |
| | C.I | (52.00-89.00) | (68.00-81.00) | | (52.00-89.00) | |
| | | 68.88-81.79 | 66.17-85.33 | | 70.58-80.29 | |
| Heart Rate | Mean ± SD | 67.00 ± 12.69 | 71.25 ± 5.12 | 0.3591 | 68.06 ± 11.27 | |
| beats/minute | Median (Min-Max) | 62.50 | 72.50 | | 66.00 | |
| | C.I | (52.00-96.00) | (64.00-76.00) | | (52.00-96.00) | |
| | | 58.94-75.06 | 63.10-79.40 | | 62.06-74.07 | |
| Hemoglobin | Mean ± SD | 15.78 ± 0.79 | 14.98 ± 1.14 | 0.2628 | 15.58 ± 0.92 | (M) 14.0-18.0 |
| (Hgb) g/dL | Median (Min-Max) | 15.65 | 15.45 | | 15.60 | (F) 12.0-16.0 |
| | C.I | (14.90-17.60) | (13.30-15.70) | | (13.30-17.60) | |
| | | 15.27-16.28 | 13.16-16.79 | | 15.08-16.07 | |
| Hematocrit | Mean ± SD | 46.21 ± 1.82 | 44.60 ± 2.60 | 0.3149 | 45.81 ± 2.08 | (M) 41.0-53.0 |
| (Hct) % | Median (Min-Max) | 46.35 | 45.30 | | 46.00 | (F) 36.0-46.0 |
| | C.I | (43.50-49.40) | (40.90-46.90) | | (40.90-49.40) | |
| | | 45.05-47.37 | 40.46-48.74 | | 44.70-46.91 | |
| White Blood Cell | Mean ± SD | 7.18 ± 1.34 | 5.68 ± 0.92 | 0.0384 | 6.80 ± 1.39 | (Age 18-20) |
| Count (WBC) | Median (Min-Max) | 7.15 | 5.60 | | 6.90 | 4.5-12.5 |
| Thousand/mm³ | C.I | (5.10-10.20) | (4.70-6.80) | | (4.70-10.20) | (Age 21-49) |
| | | 6.33-8.02 | 4.21-7.14 | | 6.06-7.54 | 4.5-11.0 |
| Red Blood Cell | Mean ± SD | 5.23 ± 0.25 | 5.04 ± 0.34 | 0.3516 | 5.18 ± 0.28 | (M) 4.60-6.20 |
| Count (RBC) | Median (Min-Max) | 5.72 | 5.08 | | 5.25 | (F) 4.20-5.40 |
| Million/mm³ | C.I | (4.88-5.72) | (4.63-5.36) | | (4.63-5.72) | |
| | | 5.07-5.39 | 4.50-5.58 | | 5.04-5.33) | |
| BUN mg/dL | Mean ± SD | 14.83 ± 3.35 | 14.75 ± 4.72 | 0.9755 | 14.81 ± 3.56 | (M) 7-18 |
| | Median (Min-Max) | 15.00 | 16.00 | | 15.00 | (F) 7-17 |
| | C.I | (9.00-20.00) | (8.00-19.00) | | (8.00-20.00) | |
| | | 12.70-16.96 | 7.24-22.26 | | 12.91-16.71 | |
| Creatinine | Mean ± SD | 1.20 ± 0.16 | 0.98 ± 0.10 | 0.0111 | 1.14 ± 0.17 | (M) 0.70-1.30 |
| mg/dL | Median (Min-Max) | 1.14 | 0.99 | | 1.10 | (F) 0.55-1.02 |
| | C.I | (1.06-1.53) | (0.88-1.08) | | (0.88-1.53) | |
| | | 1.09-1.30 | 0.83-1.14 | | 1.05-1.24 | |
| Total Bilirubin | Mean ± SD | 0.78 ± 0.23 | 0.58 ± 0.15 | 0.0802 | 0.73 ± 0.23 | 0.2-1.0 |
| mg/dL | Median (Min-Max) | 0.80 | 060 | | 0.70 | |
| | C.I | (0.50-1.10) | (0.40-0.70) | | (0.40-1.10) | |
| | | 0.63-0.92 | 0.34-0.81 | | 0.60-0.85 | |
| ALT U/L | Mean ± SD | 36.08 ± 8.87 | 23.75 ± 3.77 | 0.0020 | 33.00 ± 9.54 | 13-61 |
| | Median (Min-Max) | 34.50 | 24.00 | | 31.00 | |
| | C.I | (26.00-56.00) | (20.00-27.00) | | (20.00-56.00) | |
| | | 30.45-41.72 | 17.74-29.76 | | 27.92-38.08 | |
| AST U/L | Mean ± SD | 22.08 ± 5.65 | 12.75 ± 2.06 | 0.0003 | 19.75 ± 6.45 | 15-37 |
| | Median (Min-Max) | 22.00 | 13.00 | | 20.50 | |
| | C.I | (13.00-30.00) | (10.00-15.00) | | (10.00-30.00) | |
| | | 18.49-25.67 | 9.47-16.03 | | | |
| Fasting Blood | Mean ± SD | 88.83 ± 11.31 | 82.75 ± 8.30 | 0.2866 | 87.31 ± 10.73 | 74-99 |
| Glucose mg/dL | Median (Min-Max) | 85.50 | 83.50 | | 85.00 | |
| | C.I | (78.00-114.00) | (72.00-92.00) | | (72.00-114.00) | |
| | | 81.65-96.02 | 69.54-95.96 | | 81.60-93.03 | |

Interpretation:
All the subjects were within the age, BMI and Blood Pressure inclusion criteria. All subjects had laboratory results appropriate for study inclusion.
Any baseline differences between groups for any single parameter is not considered of great importance as all within normal limits for these blood test parameters, in addition, it is the relative change from baseline to the study time-point values that are impactful and if the difference between the two as the analytical focus.

TABLE 4
Changes in Cortisol Levels (Per-Protocol Population)

| Variable | Statistic | Time Point | Active Product | Placebo | P-Value |
|---|---|---|---|---|---|
| Serum Cortisol | Mean ± SD | Baseline (BL) | 13.09 ± 5.67 | 11.55 ± 6.93 | 0.7062 |
| | Median (Min-Max) | (Day 1 pre-exercise) | 16.00 (3.80-18.40) | 12.80 (2.00-18.60) | |
| | Mean ± SD | Final Test | 17.34 ± 7.83 | 11.80 ± 3.56 | 0.0860 |
| | Median (Min-Max) | (Day 28 pre-exercise) | 16.70 (7.30-37.00) | 12.60 (7.00-15.00) | |
| | Mean ± SD | Change from Baseline to | −4.47 ± 6.76 | −0.25 ± 4.18 | 0.1824 |
| | Median (Min-Max) | Final Test pre-exercise | −4.60 (−19.40-5.20) | −0.35 (−5.00-4.70) | |
| | p-value | | 0.0530 | 0.9124 | |
| | Mean ± SD | 10 min | 16.53 ± 7.95 | 6.88 ± 2.74 | 0.0056 |
| | Median (Min-Max) | (Day 28 post- exercise) | 16.20 (5.90-27.20) | 7.30 (3.30-9.60) | |
| | Mean ± SD | Change from Baseline to | −3.60 ± 7.12 | 4.68 ± 4.89 | 0.0366 |
| | Median (Min-Max) | 10 min post- exercise | −1.35 (−19.70-3.00) | 4.85 (−1.30-10.30) | |
| | p-value | | 0.1444 | 0.1516 | |
| | Mean ± SD | Change from Final Test | 0.74 ± 7.92 | 4.93 ± 0.85 | 0.1322 |
| | Median (Min-Max) | pre- exercise to 10 min | 0.70 (−15.10-10.50) | 5.20 (3.70-5.60) | |
| | p-value | post- exercise | 0.7744 | 0.0014 | |
| | Mean ± SD | 1 hr | 13.05 ± 5.75 | 7.08 ± 2.39 | 0.0179 |
| | Median (Min-Max) | (Day 28 post- exercise) | 11.15 (6.30-22.20) | 6.20 (5.30-10.60) | |
| | Mean ± SD | Change from Baseline to | −0.12 ± 6.71 | 4.48 ± 6.61 | 0.2891 |
| | Median (Min-Max) | 1 hr post- exercise | −0.60 (−13.80-8.40) | 4.40 (−3.30-12.40) | |
| | p-value | | 0.9561 | 0.2690 | |
| | Mean ± SD | Change from Final Test | 4.22 ± 6.46 | 4.73 ± 2.47 | 0.8360 |
| | Median (Min-Max) | pre- exercise to 1 hr post- | 4.35 (−9.20-14.80) | 4.75 (1.70-7.70) | |
| | p-value | exercise | 0.0690 | 0.0313 | |
| | Mean ± SD | 2 hr | 12.08 ± 3.54 | 14.20 ± 3.82 | 0.3808 |
| | Median (Min-Max) | (Day 28 post- exercise) | 11.70 (7.00-18.10) | 15.45 (8.70-17.20) | |
| | Mean ± SD | Change from Baseline to | 0.85 ± 5.81 | −2.65 ± 10.28 | 0.5582 |
| | Median (Min-Max) | 2 hr post- exercise | 0.10 (−8.00-10.50) | −2.65 (−15.20-9.90) | |
| | p-value | | 0.6548 | 0.6418 | |
| | Mean ± SD | Change from Final Test | 5.19 ± 6.98 | −2.40 ± 6.66 | 0.1077 |
| | Median (Min-Max) | pre- exercise to 2 hr post- | 6.25 (−5.00-18.90) | −2.30 (−10.20-5.20) | |
| | p-value | exercise | 0.0432 | 0.5234 | |
| | Mean ± SD | Change from 10 min post- | 3.48 ± 3.14 | −0.20 ± 1.76 | 0.0194 |
| | Median (Min-Max) | exercise to 1 hr post- | 4.35 (−1.80-7.00) | −0.45 (−2.00-2.10) | |
| | p-value | exercise | 0.0066 | 0.8345 | |
| | Mean ± SD | Change from 10 min post- | 4.45 ± 6.65 | −7.33 ± 5.88 | 0.0161 |
| | Median (Min-Max) | exercise to 2 hr post- | 6.60 (−4.50-11.70) | −7.50 (−13.90-(−0.40)) | |
| | p-value | exercise | 0.0635 | 0.0884 | |
| | Mean ± SD | Change from 1 hr post- | 0.97 ± 4.60 | −7.13 ± 4.58 | 0.0265 |
| | Median (Min-Max) | exercise to 2 hr min post- | 1.95 (−8.20-5.80) | −7.05 (−11.90-(−2.50)) | |
| | p-value | exercise | 0.5214 | 0.0527 | |

Interpretation:
Over the 28 day study period, those on the Study Product experienced a trend for rising Cortisol from the baseline visit to pre-stressful exercise on day 28. The rise of ~4 points for the Active Product does not appear clinically meaningful.
In general, Cortisol levels were higher in the Study Product group at 10 minutes and one hour post stressful exercise, however by the two-hour mark post stressful exercise, the drop in Cortisol was greater than that of the Placebo (cortisol recovery).

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

All United States patents and published or unpublished United States patent applications cited herein are incorporated by reference. All published foreign patents and patent applications cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

1. A method for reducing stress or anxiety, comprising administering a composition comprising inactivated, non-viable, or dead Bacillus coagulans bacteria to a subject characterized as comprising stress or anxiety.

2. The method of claim 1, wherein the composition is administered to the subject once per day.

3. The method of claim 2, wherein the composition is administered in the morning, with breakfast, or before breakfast.

4. The method of claim 2, wherein the composition is administered in the evening, with dinner, or after dinner.

5. The method of claim 1, wherein the effective amount reduces the level of cortisol in the subject.

6. The method of claim 5, wherein the level of cortisol is reduced by at least 10%.

7. The method of claim 5, wherein the level of cortisol is reduced in urine, blood, or serum of the subject.

8. The method of claim 1, wherein the subject has experienced trauma.

9. The method of claim 1, wherein the trauma comprises emotional trauma or physiological trauma.

10. The method of claim 1, wherein the subject is characterized as comprising a mood disorder.

11. The method of claim 1, wherein the subject is characterized as comprising bipolar disorder.

12. The method of claim 1, wherein the subject is characterized as comprising anxiety or depression.

13. The method of claim 1, wherein the depression is seasonal depression.

14. The method of claim 1, wherein the is characterized as comprising hypoglycemia.

15. The method of claim 1, wherein the subject is characterized as comprising post-traumatic stress disorder.

16. The method of claim 1, wherein the subject is characterized as comprising chronic pain.

17. The method of claim 1, wherein the subject is a soldier or a first responder.

18. The method of claim 1, wherein the subject does not have an infection comprising a viral, fungal, parasitic or bacterial infection.

19. The method of claim 1, wherein the composition is in the form of a tablet, a capsule, a powder, a suspension, an aqueous solution, a food, or a beverage.

20. The method of claim 1, wherein the effective amount is less than 50 mg per day.
